(19) United States
(12) Patent Application Publication
(10) Pub. No.: US 2025/0213647 A1
(43) Pub. Date: Jul. 3, 2025

(54) CHEMOKINE RECEPTORS AND ALPHA1 ADRENERGIC RECEPTORS/VASOPRESSIN RECEPTORS 1A HETEROMERS AS DRUG TARGETS FOR DISEASE

(71) Applicant: UNIVERSITY OF SOUTH FLORIDA, Tampa, FL (US)

(72) Inventor: Matthias MAJETSCHAK, Safety Harbor, FL (US)

(21) Appl. No.: 18/851,460

(22) PCT Filed: Mar. 28, 2023
(86) PCT No.: PCT/US2023/016622

§ 371 Date: Sep. 26, 2024

Related U.S. Application Data
(60) Provisional application No. 63/269,987, filed on Mar. 28, 2022.

(51) Int. Cl. A61K 38/095; A61K 31/137; A61K 45/06; A61P 29/00; A61P 35/00

(52) U.S. Cl. CPC A61K 38/095; A61K 31/137; A61K 45/06; A61P 29/00; A61P 35/00

(57) ABSTRACT

Provided are methods of modulating inflammation, modulating cancer cell trafficking, modulating chemokine receptor heteromerization, or modulating activity of a chemokine receptor by administering to a subject in need thereof a therapeutically effective amount of a modulator of an adrenergic receptor and/or an arginine vasopressin receptor. Chemokine receptor modulators can also be administered to the subject. Additionally, disclosed are compositions comprising a modulator of an adrenergic receptor and/or an arginine vasopressin receptor, and at least one cytokine receptor modulator.

### CROSS-REFERENCE TO RELATED APPLICATIONS

This application claims the benefit of priority to U.S. Provisional Patent Application No. 63/269,987 filed on Mar. 28, 2022, the disclosure of which is expressly incorporated by reference herein in its entirety.

### STATEMENT OF GOVERNMENT SUPPORT

This invention was made with government support under Grant Nos. R01GM139811and R21AI139827 awarded by the National Institutes of Health. The government has certain rights in the invention.

### BACKGROUND

The seven-transmembrane domain (7TM) receptors, of which most are G protein-coupled receptors (GPCRs), are involved in numerous aspects of human physiology and pathology. Evidence suggests that many GPCRs can form hetero-oligomeric complexes with other GPCRs, which is thought to alter their pharmacological behavior and may provide new therapeutic targets for drug development (Gomes, 2016; Farran, 2017; Dale, 2022; Ferre, 2009; Ferre, 2014; Ferre, 2020). While class C GPCR heteromers are well accepted, there is still considerable skepticism about the existence and relevance of class A GPCR heteromers

(Ferre, 2020; Franco, 2016; Lambert, 2010), and the understanding of the pharmacological behavior of such putative receptor heteromers is limited. Although criteria for the existence of GPCR heteromers in native cells and tissues have been proposed, only few of the described GPCR heteromers suffice these criteria (Gomes, 2016).

Many members of the chemokine receptor (CR) family form heteromeric complexes with α₁-adrenergic receptors (α₁-ARs) in recombinant systems, in cell lines and in primary cells and tissues of mouse, rat and human origin (Tripathi, 2015; Evans, 2016; Albee, 2017; Albee, 2021; Gao, 2018; Gao 2022; Gao, 2021; Gao, 2020; Enten, 2022). While CR heteromerization partners of α_{1B/D}-ARs (upper and lower case subscripts are used to denote endogenous and recombinant α₁-AR subtypes, respectively (Bylund, 1994) depend on the presence of α_{1B}-AR:CR heteromers to mediate chemotaxis in the monocytic cell line THP-1, both agonist and antagonist binding to α_{1B/D}-AR potently inhibited the ability of the chemokine receptor partners to mediate chemotaxis in THP-1 cells and in human monocytes (Enten, 2022). Proof of concept that α₁-AR ligands inhibit chemokine receptor-mediated chemotaxis of leukocytes in vivo, however, is missing and the molecular mechanisms underlying their inhibitory effects on chemokine receptor function are unknown.

The compositions and methods disclosed herein address these and other needs.

### SUMMARY

In accordance with the purposes of the disclosed materials and methods, as embodied and broadly described herein, the disclosed subject matter, in one aspect, relates methods of modulating inflammation, modulating cancer cell trafficking, modulating chemokine receptor heteromerization, or modulating activity of a chemokine receptor by administering to a subject in need thereof a therapeutically effective amount of a modulator of an adrenergic receptor and/or an arginine vasopressin receptor. Chemokine receptor modulators can also be administered to the subject.

Additionally, disclosed are compositions comprising a modulator of an adrenergic receptor and/or an arginine vasopressin receptor, and at least one cytokine receptor modulator.

It has previously been reported that α1-adrenoceptor (α1-AR) ligands inhibit chemokine receptor (CR) heteromerization partners of α1_{B/D}-AR. The underlying mechanisms are unknown and in vivo evidence for such effects is missing. Utilizing CCR2 and α_{B}-AR as exemplary partners, it was observed that in recombinant systems and THP-1 cells that α1_{B}-AR enhanced whereas its absence inhibited Gαi signaling of CCR2. Phenylephrine and phentolamine reduced the CCR2:α1_{B}-AR heteromerization affinity and inhibited Gαi signaling of CCR2. Phenylephrine cross-recruited β-arrestin to CCR2, and reduced expression of α1_{B/D}-AR, CR partners (CCR1/2, CXCR4) and corresponding heteromers. Phentolamine reduced CR:α1_{B/D}-AR heteromers without affecting β-arrestin recruitment or receptor expression. Phenylephrine/phentolamine prevented leukocyte infiltration mediated via CR heteromerization partners in a murine air pouch model. The α1-AR ligands inhibit leukocyte migration mediated by CR heteromerization partners in vivo and suggest interference with α1_{B}-AR:CR heteromerization as a mechanism by which CR partners are inhibited. These findings provide new insights into the pharmacology of GPCR heteromers and indicate that an agonist and antagonist at one GPCR can act as antagonists at heteromerization partners of their target receptors.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying figures, which are incorporated in and constitute a part of this specification, illustrate several aspects described below.

FIGS. 1A-1D show a ligand free α_{1b}-AR facilitates CCR2-mediated activation of Gαi1. A/B. HEK293T cells were transfected with CCR2, Gαi1-Rluc8, Gβ3, and Gγ9-GFP2 together with pcDNA3 or α_{1b}-AR as indicated in the figure. Forty-eight hours after transfection, cells were used for BRET assays and flow cytometry. FIG. 1A shows the use of flow cytometry for the detection of HA-CCR2 with anti-HA (left) and of FLAG-α_{1b}-AR with anti-FLAG (right). Red line: cells transfected with G protein biosensors, HA-CCR2 plus pcDNA3. Green line: cells transfected with G protein biosensors, HA-CCR2 plus FLAG-α_{1b}-AR. Grey area: unstained cells. FIG. 1B shows the use of BRET to measure Gαi1 activation. Cells transfected with CCR2 plus pcDNA3 (open circles) or with CCR2 plus α_{1b}-AR (grey squares) were stimulated with CCL2 and BRET measured. BRET change: BRET in the presence minus BRET in the absence of CCL2. Data are mean±SE, n=4. *:p<0.05 for CCR2 plus pcDNA3 vs. CCR2 plus α_{1b}-AR. FIG. 1C shows the use of Flow cytometry for the detection of FLAG-CCR2-Tango with anti-FLAG (left) and of HA-α_{1b}-AR with anti-HA (right). HTLA cells were transfected with FLAG-CCR2-Tango plus pcDNA3 (green line) or HA-α_{1b}-AR (red line). Grey area: unstained cells. FIG. 1D shows the use of CCR2 PRESTO-Tango β-arrestin recruitment assays. HTLA cells were transfected as in C. and stimulated with various concentrations of CCL2. Data are mean±SE, n=3. RLU, fold change: relative luminescence units (RLU) expressed as fold change in RLU over cells not exposed to CCL2.

FIGS. 2A-2F show depletion of α_{1B}-AR inhibits CCR2-mediated Gαi signaling. FIG. 2A shows THP-1 (open bars) and THP-1ADRA1B^{KO} (grey bars) cells were incubated with vehicle (ctrl.) or 10 μM forskolin (FSK) and cAMP concentrations in cell lysates measured. Data are mean±SE, n=3. FIG. 2B shows Forskolin-treated THP-1 (open bars) and THP-1ADRA1B^{KO} (grey bars) cells were stimulated with 100 nM CCL2 or CCL1 for 15 min at 37° C. and cAMP concentrations in cell lysates measured. cAMP concentrations (pmol/mL) are expressed as % inhibition of cAMP concentrations in cells not exposed to chemokines. Data are mean±SE, n=4. *:p<0.05 THP-1 vs. THP1_ADRA1B^{KO} cells. FIG. 2C shows the use of flow cytometry to analyze cell surface receptor expression in THP-1 cells after siRNA gene silencing. Gray: unstained cells; red: cells incubated with NT siRNA; green: cells incubated with α_{1B}-AR siRNA. FIG. 2D shows median fluorescence intensities (MFI) of the receptor signals in cells as in C. MFI is expressed as % of MFI in cells incubated with NT siRNA. *; p<0.05 vs.cells incubated with NT siRNA. Data are mean±SE, n=4. FIG. 2E shows THP-1 cells after incubation with NT siRNA (white bars) or α_{1B}-AR siRNA (grey bars) were incubated with vehicle (ctrl.) or 10 μM forskolin (FSK) and cAMP concentrations in cell lysates measured. Data are mean±SE, n=4. FIG. 2F shows forskolin-treated THP-1 cells, as in FIG. 2E, cells were stimulated with 100 nM CCL2 or CCL1 for 15 min at 37° C. and cAMP concentrations in cell lysates measured. cAMP concentrations (pmol/mL) are expressed as % inhibition of cAMP concentrations in cells not exposed to chemokines. Data are mean±SE, n=4. *: p<0.05 cells incubated with NT siRNA vs. cells incubated with α_{1B}-AR siRNA.

FIGS. 3A-3C show α_{1b}-AR ligands reduce the heteromerization affinity between α1_{b}-AR and CCR2. HEK293T cells were co-transfected with a fixed amount of α1_{b}-AR-R1ucII and increasing amounts of CCR2-EYFP. 48 h after transfection, cells were treated with vehicle (ctrl.), phenylephrine (PE, 10 mM) or phentolamine (PT, 10 mM) for 5 min at 37° C. before measuring BRET. FIG. 3A shows representative measurements from a titration BRET experiment. FIG. 3B shows BRETₘₐₓ values. Data are mean±SE, n=4. FIG. 3C BRET₅₀ values. Data are mean±SE, n=4. *: p<0.05 vs. ctrl.

FIGS. 4A-4H show α_{1b}-AR ligands regulate CCR2 mediated activation of Gai1 from the α_{1b}-AR:CCR2 heteromer. FIGS. 4A-4F show HEK293T cells were transfected with Gαi1-Rluc8, Gβ3, and Gγ9-GFP2 together with pcDNA3, CCR2 or α_{1b}-AR as indicated. Forty-eight hours after transfection, cells were used for BRET assays. BRET change: BRET in the presence minus BRET in the absence of receptor ligands. Data are mean±SE, n=4 or 5. *: p<0.05 vs. vehicle. FIG. 4A shows cells transfected as indicated were exposed to various concentrations of phenylephrine (PE) or phentolamine (PT). FIG. 4B shows cells transfected with CCR2 plus pcDNA3 were stimulated with various concentrations of CCL2 plus vehicle or 10 μM PE. FIG. 4C shows cells transfected with CCR2 plus α_{1b}-AR were stimulated various concentrations of CCL2 plus vehicle or 10 μM PE. FIG. 4D shows cells transfected with CCR2 plus α_{1b}-AR were stimulated with various concentrations of CCL2 plus vehicle or 1 μM norepinephrine (NE). FIG. 4E shows cells transfected with CCR2 plus pcDNA3 were stimulated various concentrations of CCL2 plus vehicle or 10 μM PT. FIG. 4F shows cells transfected with CCR2 plus α_{1b}-AR were stimulated various concentrations of CCL2 plus vehicle or 10 μM PT. FIG. 4G shows forskolin-treated THP-1 cells were stimulated with various concentrations of CCL2 in the presence of vehicle (open circles), 1 μM PE (grey squares) or 1 μM PT (grey triangles) for 15 min at 37° C. and cAMP concentrations in cell lysates measured. cAMP concentrations (pmol/mL) are expressed as % cells not exposed to CCL2. Data are mean±SE, n=4. *: p<0.05 vs. vehicle. FIG. 4H shows forskolin-treated THP-1 cells were stimulated with 10 nM CCL2 in the presence of vehicle or 100 nM NE for 15 min at 37° C. and cAMP concentrations in cell lysates measured. cAMP concentrations (pmol/mL) are expressed as % of cells not exposed to CCL2 or NE. Data are mean±SE, n=4. *: p<0.05 vs. vehicle.

FIGS. 5A-5D show activation of α_{1b}-AR enhances β-arrestin recruitment to CCR2. FIGS. 5A-5B show CCR2 PRESTO-Tango β-arrestin recruitment assays. HTLA cells were transfected with FLAG-CCR2-Tango plus pcDNA3 or HA-α_{1b}-AR, as in FIGS. 1C and 1D. RLU, fold change: relative luminescence units (RLU) expressed as fold change in RLU over cells not exposed to receptor ligands. FIG. 5A shows cells transfected as indicated were stimulated with various concentrations of PE or PT. Data are mean±SE, n=4. *:p<0.05 vs. unstimulated cells. FIG. 5B shows cells transfected with CCR2-Tango plus α_{1b}-AR were stimulated with various concentrations of CCL2 in the presence of vehicle, 1 μM PE or 1 μM PT. Data are mean±SE, n=4. *:p<0.05 vs. cells stimulated with CCL2 plus vehicle. FIGS. 5C and 5D show BRET assay for β-arrestin recruitment. HEK293T cells were transfected with CCR2-RLuc and β-arrestin-YFP with (FIG. 5D) or without (FIG. 5C) α_{1b}-AR. Cells were treated with various concentrations of CCL2 plus vehicle, 10 μM PE, PT or 100 nM INCB3284 before BRET measurements. Data are mean±SE, n=4. *:p<0.05 vs. vehicle.

FIG. 6A-6E show agonist binding to α_{1B/D}-AR induces co-internalization α_{1B/D}-AR and their chemokine receptor heteromerization partners. FIG. 6A shows THP-1 cells were incubated with vehicle (ctrl., top), 10 μM phenylephrine (PE, center) or 10 μM phentolamine (PT, bottom) for 30 min at 37° C. and cell surface expression of individual receptors visualized by PLA. Images show merged 4′,6-diamidino-2-phenylindole (DAPI, nuclear counterstain) and PLA signals (red, λexcitation/emission 598/634 nm) acquired from z-stack images (n=10; thickness 0.5 μm, bottom to top) and are representative of n=3 independent experiments. Scale bar, 10 μm. FIGS. 6B-6G show quantification of PLA signals for α_{1B}-AR (FIG. 6B), α_{1D}-AR (FIG. 6C), CCR1 (FIG. 6D), CCR2 (FIG. 6E), CCR8 (FIG. 6F) and CXCR4 (FIG. 6G) from n=3 experiments. Data are mean±SE, n=3. *:p<0.05 vs. ctrl.

FIGS. 7A-7B show agonist binding to α_{1B/D}-AR enhances CCL2-induced internalization of CCR2. FIG. 7A shows THP-1 cells were incubated with 100 nM CCL2 (left) or 100 nM CCL2 plus 1 μM phenylephrine (PE, right) at 37° C. for various time periods (0-45 min, color coded as indicated). Cell surface CCR2 expression was analyzed by flow cytometry. Grey areas: unstained cells. FIG. 7B shows quantification of CCR2 expression from n=4 independent experiments, as in A. Open circles: cells stimulated with CCL2. Grey squares: cells stimulated with CCL2 plus PE. CCR2 expression (% ctrl.): median fluorescence intensity (MFI) in percent of the MFI measured at 0 min (=100%). Data are mean±SE. *:p<0.05 CCL2 vs. CCL2 plus PE.

FIGS. 8A-8G show α_{1B/D}-AR ligands interfere with heteromerization between α_{1B/D}-AR and their chemokine receptor partners. FIG. 8A shows THP-1 cells were incubated with vehicle (ctrl., top), 10 μM phenylephrine (PE, center) or 10 μM phentolamine (PT, bottom) for 30 min at 37° C. and cell surface expression of receptor-receptor interactions visualized by PLA. Images show merged 4′,6-diamidino-2-phenylindole (DAPI, nuclear counterstain) and PLA signals (red, λexcitation/emission 598/634 nm) acquired from z-stack images (n=10; thickness 0.5 μm, bottom to top) and are representative of n=3 independent experiments. Scale bar, 10 μm. Quantification of PLA signals for heteromers between α_{1B}-AR and CCR1 (FIG. 8B), λ_{1B}-AR and CCR2 (FIG. 8C), α_{1B}-AR and CXCR4 (FIG. 8D), α_{1D}-AR and CCR1 (FIG. 8E), α_{1D}-AR and CCR2 (FIG. 8F) and α_{1D}-AR and CXCR4 (FIG. 8G) from n=3 experiments. Data are mean±SE, n=3. *:p<0.05 vs. ctrl.

FIGS. 9A-9D show α₁-AR ligands inhibit chemotaxis of leukocytes mediated by chemokine receptor heteromerization partners of α_{1B/D}-ARs in vivo. FIG. 9A shows human CCL2 activates mouse and human CCR2. HEK293T cells were transfected with Gαi1-Rluc8, Gβ3, and Gγ9-GFP2 together with human or mouse CCR2. Forty-eight hours after transfection, cells were stimulated with human CCL2 and BRET measured. BRET change: BRET in the presence minus BRET in the absence human CCL2. Data are mean±SE, n=3. FIGS. 9B-9D show analysis of leukocyte infiltration into dorsal air pouches of C57BL/6 mice. Mice received injections of vehicle (−) or 2.5 mg/kg LPS (FIG. 9B), 6 μg CCL2 (FIG. 9C), or 2 μg CXCL12 (FIG. 9D) plus vehicle or various doses of phenylephrine and phentolamine, as indicated, into the air pouches. Twenty-four hours later, air pouch cells were harvested and analyzed by flow cytometry. Leukocytes (% ctrl.): Number of CD45 positive cells/mL in % of the number of cells/mL after vehicle injection (−). Data are mean±SE, n=3-4 mice/condition. *:p<0.05 vs. injection of the chemotactic agent plus vehicle (+).

FIG. 10 shows HEK293T cells were transfected with AVPRIA-RLuc plus each CR-YFP in triplicate. Net BRET signals are mean±SD. Cells transfected with AVPR1A-RLuc and mGlu1R-YFP at various acceptor:donor ratios served as nonspecific controls; non-specific BRET signals were analyzed by linear regression analysis. The black line shows the regression line, dashed lines indicate 99% prediction bands. The grey area indicates the expected distribution of non-specific BRET signals. The graph represents one of three screening experiments. Right: Results from three screening experiments. n/3=number of positive BRET signals (above the 99% prediction band for non-specific interactions) out of 3 independent BRET screening experiments.

FIGS. 11A-11E show HEK293T cells were transfected with a fixed amount of AVPR1A-RLuc and with increasing amounts of CR-YFP or mGlu1R-YFP (=control), as indicated. Figures show saturation BRET signals representative of 3 independent experiments per combination.

FIGS. 12A-12B show representative PLA images for the detection of individual receptors (FIG. 12A) and receptor-receptor interactions (FIG. 12B) in THP-1 cells. PLA was performed as we described previously. Images show merged 4′,6-diamidino-2-phenylindole (DAPI, nuclear counterstain) and PLA signals (red, lexcitation/emission 598/634 nm) acquired from z-stack images (n=10; thickness 0.5 μm, bottom to top) and are representative of n=3 independent experiments.

FIGS. 13A-13C show ligands of AVPR1A regulate chemotaxis mediated via CR heteromerization partners of AVPR1A. CI (%):Chemotactic index in % of cells not exposed to arginine vasopressin (AVP)/Conivaptan (mean±SE). THP-1 cells were exposed to various concentrations AVP (FIG. 13A, 13B) or Conivaptan (FIG. 13C) and chemotaxis toward CCL23 (10 nM), CCL2 (10 nM), CCL1 (1 μM), CXCL12 (100 nM) or CXCL8 (10 nM) tested, as indicated. N=3-4.

FIGS. 14A-14E show VRET screening to identify chemokine receptor heteromerization partners of α_{1a/b/d}-adrenergic receptors. HEK293T cells were transfected with α_{1a/b/d}-AR-Rluc plus each CR-YFP in triplicate. YFP fluorescence and luminescence were read as described in Methods. Net BRET (528 nm/460 nm) was plotted against YFP fluorescence/luminescence (YFP/lum). Net BRET signals are mean±SD. Cells transfected with α_{1a/b/d}-AR-Rluc and YFP or mGlu₁R-YFP at various acceptor:donor ratios served as non-specific controls; non-specific BRET signals were analyzed by linear regression analysis. The black line shows the regression line, dashed lines indicate 99% prediction bands. The grey area indicates the expected distribution of non-specific BRET signals. BRET signals above the 99% prediction band for non-specific interactions were considered as positive signals for interactions between CRs (colored symbols) and α_{1a/b/d}-AR. The graphs represent one of three screening experiments for interactions between CRs and α₁ₐ-AR (FIGS. 14A, 14B), α_{1b}-AR (FIG. 14C) or α_{1d}-AR (FIG. 14D, 14E). To minimize type I error (false positive identification), mean BRET signals corresponding to heteromeric complexes between α_{1a/b/d}-AR-Rluc and CR-YFP above the 99% prediction band were considered for non-specific interactions (grey areas in FIGS. 14A-14E) as positive signals.

FIGS. 15A-15P show the results of saturation BRET experiments. These experiments were performed to confirm the results from the BRET screening experiments. Saturation BRET confirmed chemokine receptor heteromerization partners of α1_{a/b/d}-adrenergic receptors. For each α1-AR subtype, several CRs were randomly selected that showed positive BRET signals and at least one CR with negative BRET signals in the screening experiments (FIGS. 15A-15P). HEK293T cells were transfected with a fixed amount of α1_{a/b/d}-RLuc and with increasing amounts of CR-YFP or YFP. Figures show saturation BRET signals representative of n=3 independent experiments per receptor-receptor combination. YFP fluorescence and luminescence were read as described in Methods. Net BRET (528 nm/460 nm) was plotted against YFP fluorescence/luminescence (YFP/Lum). Saturation BRET between α1ₐ-AR and CCR1 (FIG. 15A), CXCR4 and YFP (FIG. 15B), CXCR5 (FIG. 15C), XCR1 (FIG. 15D) or ACKR4 (FIG. 15E). Saturation BRET between α1_{b}-AR and CCR1 and YFP (FIG. 15F), CCR2 (FIG. 15G), CCR4 (FIG. 15H), CCR10 (FIG. 15I), CXCR4 (FIG. 15J, ACKR1 (FIG. 15K), ACKR2 (FIG. 15L) or CCR8 (FIG. 15M). Saturation BRET between α1_{d}-AR and CCR6 and YFP (FIG. 15N), CXCR2 (FIG. 150) or CCR9 (FIG. 15P).

FIGS. 16A-16D show chemokine receptor:α1_{A/B/D}-adrenergic receptor heteromers that are detectable in THP-1 cells and in human monocytes. Representative PLA images for the detection of individual receptors (FIGS. 16A, 16C) and receptor-receptor interactions (FIGS. 16B, 16D) in THP-1 cells (FIGS. 16A, 16B) and freshly isolated monocytes (FIGS. 16C, 16D). Images show merged 4′,6-diamidino-2-phenylindole (DAPI, nuclear counterstain) and PLA signals (red, λ_{excitation/emission} 598/634 nm) acquired from z-stack images (n=10; thickness 0.5 μm, bottom to top) and are representative of n=3 independent experiments. As controls, cells were incubated with IgG (FIGS. 16A, 16C) or with a combination of IgG and anti-CR (FIGS. 16B, 16D). (FIGS. 16A, 16B) Scale bars, 10 μm; (FIGS. 16C, 16D) Scale bars, 5 μm.

FIGS. 17A-17J show ligands of α₁-adrenergic receptors inhibit chemotaxis mediated via chemokine receptor heteromerization partners of α17_{/D}-adrenergic receptors. CI: Chemotactic Index (mean±SE). THP-1 cells (FIG. 17A) or freshly isolated human monocytes (FIG. 17B) were exposed to various concentrations phenylephrine (PE) and chemotaxis toward CCL23 (10 nM), CCL2 (10 nM), CCL1 (1 nM) or CXCL12 (100 nM) tested. CI (%): Chemotactic index in % of cells not exposed to PE. N=3-4 independent experiments. FIG. 17C shows THP-1 cells were exposed to various concentrations of phentolamine and chemotaxis toward CCL23 (10 nM), CCL2 (10 nM), CCL1 (1 nM) or CXCL12 (100 nM) tested. CI (%): Chemotactic index in % of cells not exposed to phentolamine. N=3 independent experiments. THP-1 cells were exposed to various concentrations of 5-Methylurapidil, L-786314 or BMY7378 and chemotaxis toward CCL23 (10 nM, FIG. 17D), CCL2 (10 nM, FIG. 17E), or CXCL12 (100 nM, FIG. 17F) tested. CI (%):Chemotactic index in % of cells not exposed to inhibitors. N=3 independent experiments. Chemotactic dose-responses for CCL23 (FIG. 17G, n=3), CCL2 (FIG. 17H, n=6) and CXCL12 (FIG. 17I, n=3) in THP-1 cells exposed to 10 nM phenylephrine (PE), 10 nM phentolamine or vehicle. *:p<0.05 for PE vs. vehicle; *:p<0.05 for phentolamine vs. vehicle (2-way ANOVA with Dunnett's multiple comparisons test). FIG. 17J shows radioligand competition binding assays with crude membrane preparations from THP-1 cells exposed to vehicle, 10 nM PE or 10 nM phentolamine. Specific [¹²⁵I]-CCL2 binding (%): (cpm-non-specific cpm at bottom plateau)/(cpm in the absence of CCL2-non-specific cpm)×100. Data are mean±SE from n=3 independent experiments performed in duplicates.

FIG. 18 shows that depletion of α1_{8/D}-adrenergic receptors by siRNA gene silencing does not affect chemokine receptor expression. THP-1 cells were incubated with non-targeting (NT), α1_{B}-AR or α1_{D}-AR siRNA and receptor expression measured by PLA. Images show merged DAPI/PLA signals for the detection of individual receptors, as indicated, and are representative of n=3 independent experiments. Scale bars, 10 μm. Bottom row: Quantification of the number of PLA signals per cell for individual receptors in THP-1 cells after NT siRNA (ctrl., black bars), α1_{B}-AR siRNA (light grey bars), or α1_{D}-AR siRNA (dark grey bars) treatment (n=3). Data (mean±SE) are expressed as a percent (%) of cells treated with NT siRNA. *:p<0.05 vs. cells treated with NT siRNA (1-way ANOVA with Dunnett's multiple comparisons test). Incubation of THP-1 cells with α1_{B/D}-AR siRNA selectively reduced PLA signals for the target receptor without affecting PLA signals for other receptors that were studied. As compared with cells incubated with NT siRNA, PLA signals for α1_{B}-AR were reduced by 57±7% and PLA signals for α1_{D}-AR were undetectable after incubation with the corresponding siRNA.

FIG. 19 shows chemokine receptor:α1_{8/D}-adrenergic receptor heteromers after α1_{8/D}-adrenergic receptor siRNA knockdown. This includes representative images for the visualization of receptor-receptor interactions after incubation of cells with non-targeting (NT) and α1_{B/D}-AR siRNA, and the quantification of PLA signals from three independent experiments. THP-1 cells were incubated with non-targeting (NT), α1_{B}-AR or α1_{D}-AR siRNA (same cells as in FIG. 5) and receptor-receptor interactions measured by PLA. Images show merged DAPI/PLA signals for the detection of receptor-receptor interactions, as indicated, and are representative of n=3 independent experiments. Scale bars, 10 μm. Bottom row: Quantification of the number of PLA signals per cell for receptor-receptor interactions in THP-1 cells after NT siRNA (ctrl., black bars), α1_{B}-AR siRNA (light grey bars), or α1_{D}-AR siRNA (dark grey bars) treatment (n=3). Data are expressed as a percent (%) of cells treated with NT siRNA. *:p<0.05 vs. cells treated with NT siRNA (1-way ANOVA with Dunnett's multiple comparisons test). PLA signals corresponding to CCR1:α1_{B/D}-AR heteromers were reduced consistent with the degree of α1_{B/D}-AR depletion after siRNA treatment. In contrast, incubation of cells with α1_{B}-AR siRNA reduced PLA signals for CCR2:α1_{B}-AR and CXCR4:α1_{B}-AR interactions as well as PLA signals for CCR2:α1_{D}-AR and CXCR4:α1_{D}-AR interactions, as compared with cells incubated with NT siRNA. Because α1_{B}-AR is known to form heteromeric complexes with α1_{A}-AR and α1_{D}-AR in recombinant systems and in human vascular smooth muscle (Uberti, 2003; Albee, 2021; Hague, 2006), findings suggest that CCR1 primarily interacts with α1_{B/D}-AR protomers or homodimers, whereas CCR2 and CXCR4 form higher-order hetero-oligomeric complexes with the α1_{B}-AR:α1_{D}-AR heteromer. Although the existence of higher-order hetero-oligomeric GPCR complexes composed of more than two GPCR protomers in native cells or tissues has as yet not been unequivocally confirmed due to methodological limitations, a class A hetero-oligomeric GPCR complex composed of four distinct protomers can be formed and exhibits pharmacological properties distinct from the individual protomers in a recombinant system (Gao, 2021). The chemotactic dose-response curves for each chemokine in THP-1 cells after incubation with NT and α1_{B}-AR siRNA are shown in FIGS. 20A-20D, and after incubation with NT and α1_{D}-AR siRNA in FIG. 20E-20H, respectively.

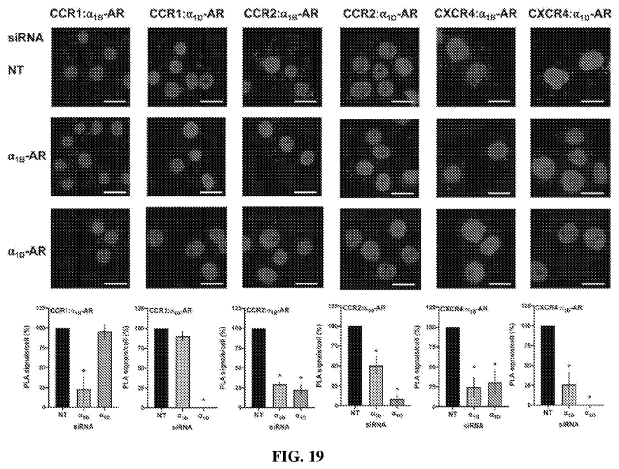

FIGS. 20A-20H show α1_{8/D}-adrenergic receptor siRNA knockdown partially inhibits chemotaxis mediated via chemokine receptor heteromerization partners of α1_{b/d}-adrenergic receptors. THP-1 cells were treated as in FIGS. 19 and 20A-20H and chemotaxis toward CCL23 (FIG. 20A, 20E), CCL2 (FIG. 20B, 20F), CCL1 (FIG. 20C, 20G) and CXCL12 (FIG. 20D, 20H) tested. CI: Chemotactic index (mean±SE, n=3-4/condition). *:p<0.05 vs. cells incubated with non-targeting (NT) siRNA (2-way ANOVA with Dunnett's multiple comparisons test). As compared with cells incubated with NT siRNA, incubation of cells with α1_{B}-AR siRNA partially reduced chemotactic activity of the CCR1, CCR2 and CXCR4 agonists (FIGS. 7A/B/D). Incubation of cells with α1_{D}-AR siRNA also inhibited the chemotactic activity of the CCR2 and CXCR4 agonists (FIGS. 7F/H) but did not affect CCR1 (FIG. 7E), when compared with cells incubated with NT siRNA. Consistent with the lack of effect of α1-AR ligands on CCR8 function, CCR8-mediated chemotaxis was not affected by α1_{B/D}-AR knockdown. The findings that α1_{B}-AR as well as α1_{D}-AR knockdown partially inhibited CCR2 and CXCR4-mediated chemotaxis are consistent with our observations indicating that these CRs hetero-oligomerize with the α1_{B}-AR:α1_{D}-AR heteromer. Similarly, the findings that α1_{B}-AR knockdown inhibited CCR1-mediated chemotaxis whereas α1_{D}-AR knockdown was ineffective further support the assumption that CCR1:α1_{B}-AR and CCR1:α1_{D}-AR heteromers exist as separate and independent entities. Although the function of CCR1:α1_{D}-AR heteromers remain to be determined, our findings are consistent with the notion that the presence of α1_{B}-AR or α1_{B/D}-AR heteromers facilitates normal function of their CR heteromerization partners. To consolidate these findings, we utilized CRISPR/Cas 9 gene editing to generate a THP-1 cell line that lacks α1_{B}-AR.

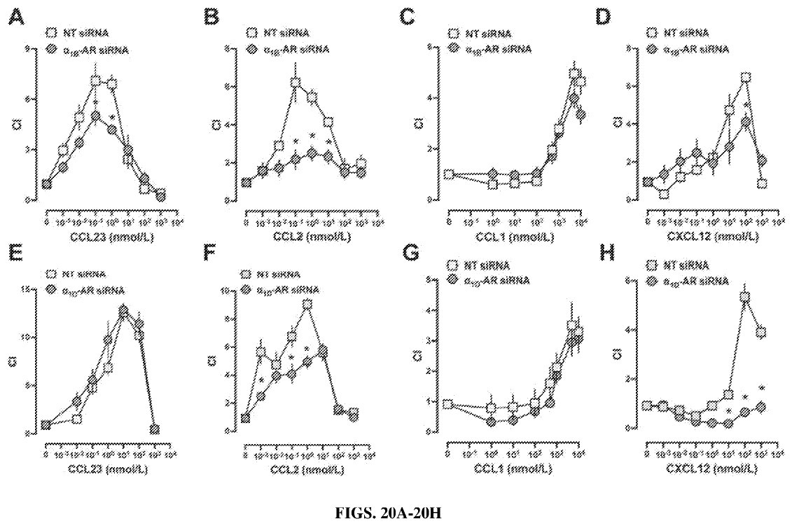

FIGS. 21A-21H show chemokine receptor heteromerization partners of α1_{8/D}-adrenergic receptors require α1_{8/D}-adrenergic receptors to mediate chemotactic responses. FIGS. 21A,21B show CRISPR/Cas 9 gene editing to generate a THP-1 cell line that lacks α1_{B}-AR, designated THP-1_ADRA1B^{KO}. FIG. 21A shows T7 surveyor assay to confirm the gene modification in the targeted region of ADRA1B. Images from agarose gel electrophoresis for the detection of PCR amplified ADRA1B genomic DNA before (top) and after T7 endonuclease I digestion (bottom) from a wild type THP-1 cell clone (Lane 1, ctrl. (wt)) and from puromycin selected THP-1 cell clones that were transduced with lentivirus encoding sgRNA targeting α1_{B}-AR and Cas9 (lanes 2-6). Lane 7: DNA ladder. The PCR amplified ADRA1B genomic DNA before and after T7 endonuclease I digestion are from a wild type THP-1 cell clone (Lane 1) and from puromycin selected THP-1 cell clones that were transduced with lentivirus encoding sgRNA targeting α1_{B}-AR and Cas9 (lanes 2-6). FIG. 21B shows a scheme depicting the modified genomic region of ADRA1B in THP-1_ADRA1B^{KO} cells. FIG. 21C shows detection of individual receptors in a THP-1 wild type clone (ctrl., top) and in THP-1_ADRA1B^{KO} cells (bottom) by PLA. Images show merged DAPI/PLA signals and are representative of n=3 independent experiments. Scale bars, 10 μm. FIG. 21D shows quantification of PLA signals per cell for the detection of individual receptors in THP-1_ADRA1B^{KO} cells. Data (mean±SE) are expressed as a percent (%) of a wild type THP-1 cell clone (% ctrl.). *: p<0.05 vs. ctrl (unpaired Student's t-test). Chemotaxis of THP-1_ADRA1B^{KO} cells and wt THP-1 cells toward CCL23 (FIG. 21E), CCL2 (FIG. 21F), CCL1 (FIG. 21G) and CXCL12 (FIG. 21H). CI: Chemotactic index (mean mean±SE, n=3-4 independent experiments). *:p<0.05 for THP-1_ADRA1B^{KO} cells vs. wt THP-1 cells (2-way ANOVA with Dunnett's multiple comparisons test).

FIG. 22 shows results from three independent BRET screening experiments to identify chemokine receptor heteromerization partners of α_{1a/b/d/}-adrenergic receptors. BRET was performed as in FIGS. 15A-15E. BRET+: number of BRET signals above the 99% prediction band for no-specific interactions out of 3 independent experiments. BRET+signals in 3/3, 2/3, 1/3 and 0/3 experiments are highlighted in red, orange, tallow and grey, respectively. Further, this figure summarizes the results from three independent screening experiments for each α₁-AR subtype. Positive BRET signals were observed for interactions between twenty of the 23 members of the CR family with at least one of the α₁-AR subtypes in 3 of 3 screening experiments (19, 7 and 10 positive signals in all screening experiments for interactions between CRs and α₁ₐ-AR, α_{1b}-AR and α_{1d}-AR, respectively). Chemokine (X-C motif) receptor 1 (XCR1) was the only CR for which BRET signals for interactions with α_{1a/b/d}-ARs were below the 99% prediction band for non-specific interactions in all screening experiments. The finding that BRET signals for interactions between ACKR3 and α_{1a/b}-ARs were positive in only 1 of the 3 screening experiments and BRET signals for interactions between ACKR3 and α_{1d}-AR were negative could be explained by previous observations, which suggested that α1-ARs preferentially hetero-oligomerize with ACKR3 within the CXCR4:ACKR3 heterodimer (Albee, 2017).

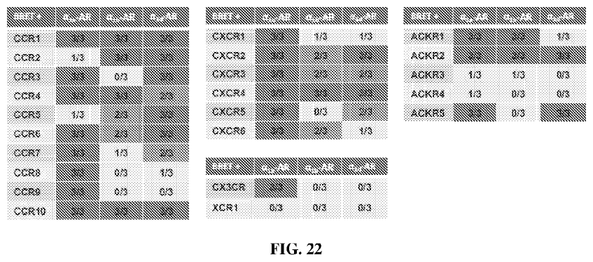

FIGS. 23A-23F show chemotactic responses of THP-1 cells. CI: chemotactic index (mean±SE). FIGS. 23A-23D show chemotactic dose-responses for CCL23 (FIG. 23A), CCL2 (FIG. 23B), CCL1 (FIG. 23C) and CXCL12 (FIG. 23D) in THP-1 cells. FIG. 23E shows THP-1 cells exposed to various concentrations of CCR2/3/5 inhibitors (INCB3344/SB-328437/Maraviroc) alone or in combination, and chemotaxis toward CCL2 (10 nM) was tested. CI (%): chemotactic index in % of cells not exposed to any of the inhibitors. N=3 independent experiments/condition. FIG. 23F shows ligands of α₁-Ars do not induce chemotaxis in THP-1 cells. Migration of THP-1 cells toward various concentrations of α₁-AR ligands were tested as indicated. N=3 independent experiments.

### DETAILED DESCRIPTION

The following description of the disclosure is provided as an enabling teaching of the disclosure in its best, currently known embodiments. Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.”

As used in the specification and the appended claims, the singular forms “a,” “an” and “the” include plural referents unless the context clearly dictates otherwise. Thus, for example, reference to “a compound”, “a composition”, or “a disorder”, includes, but is not limited to, two or more such compounds, compositions, or disorders, and the like.

The term “patient” preferably refers to a human in need of treatment with for any purpose, and more preferably a human in need of such a treatment to treat cancer or inflammation, or a precancerous condition or lesion. However, the term “patient” can also refer to non-human animals, such as dogs, cats, horses, cows, pigs, sheep and non-human primates, among others, that are in need of treatment with an anti-cancer agent or treatment.

By “reduce” or other forms of the word, such as “reducing” or “reduction,” is meant lowering of an event or characteristic (e.g., tumor growth or inflammation). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control (e.g., an untreated tumor).

By “increase” or other forms of the word, such as “increasing”, is meant raising the frequency or severity of an event or characteristic (e.g., tumor growth or inflammation). It is understood that this is typically in relation to some standard or expected value, in other words it is relative, but that it is not always necessary for the standard or relative value to be referred to. For example, “increases CR activity” means increasing activity of a CR as relative to a standard or a control.

By “modulate” is meant to change, either in terms of frequency, severity, intensity, or activity. For example, receptors disclosed herein can be modulated, that is their activity can be changed (made more or less active). A “modulator” is a substance that change the outcome of a physical, chemical, or biological process. Thus, a modulator can be an activator or inhibitor.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

### Compositions and Methods

Provided herein is a method of modulating inflammation comprising administering to a subject in need thereof a therapeutically effective amount of a modulator of an adrenergic receptor and/or an arginine vasopressin receptor. Additionally, disclosed herein is a method of modulating cancer cell trafficking comprising administering to a subject in need thereof a therapeutically effective amount of a modulator of an adrenergic receptor and/or an arginine vasopressin receptor.

Still further, disclosed herein is a method of modulating chemokine receptor heteromerization comprising administering to a subject in need thereof a therapeutically effective amount of a modulator of an adrenergic receptor and/or an arginine vasopressin receptor. Further still, disclosed herein is a method of modulating activity of a chemokine receptor comprising administering to a subject in need thereof a therapeutically effective amount of a modulator of an adrenergic receptor and/or an arginine vasopressin receptor. Examples of chemokine receptors that can be modulated or that have their heteromerization modulated include, for example, CCR1, CCR2, CCR3, CCR4, CCR5, CCR6, CCR7, CCR8, CCR9, CCR10, CXCR1, CXCR2, CXCR3, CXCR4, CXCR5, CXCR6, ACKR1, ACKR2, ACKR3, ACKR4, ACKR5, or CX3CR.

The adrenergic receptor for which a modulator thereof can be used can be alpha1-adregeneric receptor. The arginine vasopressin receptor for which a modulator can be used can be arginine vasopressin receptor 1A.

Adrenergic Receptor antagonist that can be used in the disclosed methods include esmolol, betaxolol, metoprolol, dapiprazole, atenolol, alfuzosin, mirtazapine, timolol, profenamine, prazosin, sotalol, carteolol, propranolol, doxazosin, labetalol, bisoprolol, phentolamine, nicergoline, tamsulosin, tolazoline, alprenolol, quinidine, phenoxybenzamine, pindolol, ergoloid mesylate, carvedilol, bretylium, terazosin, acebutolol, nadolol, levobunolol, metipranolol, bevantolo, practolol, penbutolol, yohimbine, oxprenolol, 1-benzylimidazole, celiprolol, silodosin, esmirtazapine, bufuralol, bopindolol, bupranolol, lurasidone, indoramin, indenolol, ifenprodil, befunolol, arotinolol, moxisylyte, trimazosin, atipamezole, talinolol, naftopidil, landiolol, bunazosin, idazoxan, urapidil, bucindolol, dihydroergocristine, cloranolol, mepindolol, epanololl, tertatolol, nebivolol, esatenolol, asenapine, propafenone, levobetaxolol, buflolmedil, dutasteride, finasteride, ziprasidone, thioridazine, flupentixol, promazine, trazodone, risperidone, propiomazine, trifluoperazine, nefazodone, methotrimeprazine, dronedarone, nicardipine, paliperidone, quetiapine, clozapine, aripiprazole, olanzapine, droperidol, zuclopenthixol, amitriptyline, doxepin, imipramine, nortriptyline, amoxapine, trimipramine, chlorpromazine, acepromazine, thioproperaine, iloperidone, niguldipine, verapamil, pizotifen, propiverine, periciazine, brexpiprazole, bromocriptine, anisodamine, ergotamine, aripiprazole lauroxil, dexpropranolol, guanadrel, guanethidine, orm-12741, dihydroergotoxine, viloxazine, and any combination thereof.

Adrenergic Receptor agonist that can be used in the disclosed methods include droxidopa, pseudoephedrine, ephedrine, dipivefrin, midodrine, isoetharine, norepinephrine, phenylephrine, phenylpropanolamine, brimonidine, clonidine, metaraminol, guanabenz, dexmedetomidine, epinephrine, tizanidine, methoxamine, orciprenaline, dobutamine, ritodrine, terbutaline, bitolterol, oxymetazoline, salmeterol, apraclonidine, mehyldopa, formoterol, salbutamol, guanfacine, isoprenaline, arbutamine, arformoterol, fenoterol, pirbuterol, mephentermine, procaterol, clenbuterol, nebivolol, lofexidine, amibegro, nylidrin, solabegron, naphazoline, mirabegron, adrafinil, isoxsuprine, hexoprenaline, etilefrine, befunolol, olodaterol, cirazoline, synephrine, racepinephrine, amitraz, medetomidine, xylazine, ractopamine, romofidine, detomidine, rilmenidine, ritobegron, tulobuterol, dopexamine, higenamine, reproterol, octopamine, norfenefrine, oxyfedrine, rimiterol, methoxyphenamine, tretoquinol, prenalterol, xamoterol, ephedra sinica root, cl-methylephedrine, xylometazoline, pergolide, bromocriptine, metamfetamine, moxonidine, phendimetrozine, ergometrine, isometheptene, tetryzoline, etomidate, bambuterol, indacaterol, vilanterol, celiprolol, levosalbutamol, doxofylline, protokylol, etafedrine, bethanidine, abediterol, PF-00610355, anisodamine, hydroxyamphetamine, benzaphetamine, 4-methoxyamphetamine, droxidopa, and any combination thereof.

Arginine Vasopressin Receptor 1A antagonists that can be used in the disclosed methods include conivaptan, tolvaptan, lixivaptan, satavaptan, relcovaptan, nelivaptan, lixivaptan, mozavaptan, somatostatin, balovaptan, and any combination thereof.

Arginine Vasopressin Receptor 1A agonists that can be used in the disclosed methods include selepressin, terlipressin, and combinations thereof.

In specific examples, the modulator of an adrenergic receptor can be phenylephrine, phentolamine, norepinephrine 5-methylurapidil, L-786314, BMY7378, or any combination thereof.

Further, the disclosed methods can additional comprise administering a therapeutically effective amount of a cytokine receptor modulator. Examples of cytokine receptor modulators that can be used herein include maraviroc, plerixafor, vicriviroc, aplaviroc, BX471, CP-481,715, MK-0812, T-487 (AMG-487), ZK-756326, IL-8, VUF 11207, Rh-SDF1α, AMD3100, and any combination thereof.

When a cytokine receptor modulator is used in combination with an adrenergic receptor and/or an arginine vasopressin receptor, the therapeutically effective amount of the cytokine receptor modulator can be less than a therapeutically effective amount of cytokine receptor modulator when no modulator of an adrenergic receptor or an arginine vasopressin receptor is administered to the subject. That is, because the adrenergic receptor and/or an arginine vasopressin receptor affect heteromerization with a cytokine receptor, and thus the activity of the cytokine receptor, the amount of cytokine receptor modulator used can be less than normally needed for a therapeutically effective amount. The amount less can be 10%, 20%, 30%, 40%, 50%, 60%, 70%, or 80% less.

The inflammatory conditions that can be treated by the disclosed methods include infection, trauma, autoimmune diseases, cardiovascular disease, and cancer.

Examples of “autoimmune disease” treatable by the disclosed methods include a set of diseases, disorders, or conditions resulting from an adaptive immune response (T cell and/or B cell response) against the host organism. In such conditions, either by way of mutation or other underlying cause, the host T cells and/or B cells and/or antibodies are no longer able to distinguish host cells from non-self-antigens and attack host cells bearing an antigen for which they are specific. Examples of autoimmune diseases include, but are not limited to graft versus host disease, transplant rejection, Achalasia, Acute disseminated encephalomyelitis, Acute motor axonal neuropathy, Addison's disease, Adiposis dolorosa, Adult Still's disease, Agammaglobulinemia, Alopecia areata, Alzheimer's disease, Amyloidosis, Ankylosing spondylitis, Anti-GBM/Anti-TBM nephritis, Antiphospholipid syndrome, Aplastic anemia, Autoimmune angioedema, Autoimmune dysautonomia, Autoimmune encephalomyelitis, Autoimmune enteropathy, Autoimmune hemolytic anemia, Autoimmune hepatitis, Autoimmune inner ear disease (AIED), Autoimmune myocarditis, Autoimmune oophoritis, Autoimmune orchitis, Autoimmune pancreatitis, Autoimmune polyendocrine syndrome, Autoimmune retinopathy, Autoimmune urticaria, Axonal & neuronal neuropathy (AMAN), Baló disease, Behcet's disease, Benign mucosal pemphigoid, Bickerstaff's encephalitis, Bullous pemphigoid, Castleman disease (CD), Celiac disease, Chagas disease, Chronic fatigue syndrome, Chronic inflammatory demyelinating polyneuropathy (CIDP), Chronic recurrent multifocal osteomyelitis (CRMO), Churg-Strauss Syndrome (CSS), Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Diabetes mellitus type 1, Discoid lupus, Dressler's syndrome, Endometriosis, Enthesitis, Eosinophilic esophagitis (EoE), Eosinophilic fasciitis, Erythema nodosum, Essential mixed cryoglobulinemia, Evans syndrome, Felty syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's encephalopathy, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenia purpura (ITP), Inclusion body myositis (IBM), Interstitial cystitis (IC), Inflamatory Bowel Disease (IBD), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus nephritis, Lupus vasculitis, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Ord's thyroiditis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Rheumatoid vasculitis, Sarcoidosis, Schmidt syndrome, Schnitzler syndrome, Scleritis, Scleroderma, Sjögren's syndrome, Sperm & testicular autoimmunity, Stiff person syndrome (SPS), Susac's syndrome, Sydenham chorea, Sympathetic ophthalmia (SO), Systemic Lupus Erythematosus, Systemic scleroderma, Takayasu's arteritis, Temporal arteritis/Giant cell arteritis, Thrombocytopenia purpura (TTP), Tolosa-Hunt syndrome (THS), Transverse myelitis, Type 1 diabetes, Ulcerative colitis (UC), Undifferentiated connective tissue disease (UCTD), Urticaria, Urticarial vasculitis, Uveitis, Vasculitis, Vitiligo, Vogt-Koyanagi-Harada Disease, and Wegener's granulomatosis (or Granulomatosis with Polyangiitis (GPA)).

Examples of cardiovascular disease treatable by the disclosed methods include coronary artery disease, abnormal left ventricular end-diastolic pressure disease (LVEDP), pulmonary hypertension and subcategories thereof, heart failure (HF), among others as discussed herein. In the context of cardiovascular and respiratory systems, examples of diseases and conditions to which such metrics can relate include, for example: (i) heart failure (e.g., left-side or right-side heart failure; heart failure with preserved ejection fraction (HFpEF)), (ii) coronary artery disease (CAD), (iii) various forms of pulmonary hypertension (PH) including without limitation pulmonary arterial hypertension (PAH), (iv) abnormal left ventricular ejection fraction (LVEF), and various other diseases or conditions. An example indicator of certain forms of heart failure is the presence or non-presence of elevated or abnormal left-ventricular end-diastolic pressure (LVEDP). An example indicator of certain forms of pulmonary hypertension is the presence or non-presence of elevated or abnormal mean pulmonary arterial pressure (mPAP).

Examples of cancers that can be treated by the disclosed methods include cancer and/or tumors of the anus, bile duct, bladder, bone, bone marrow, bowel (including colon and rectum), breast, eye, gall bladder, kidney, mouth, larynx, esophagus, stomach, testis, cervix, head, neck, ovary, lung, mesothelioma, neuroendocrine, penis, skin, spinal cord, thyroid, vagina, vulva, uterus, liver, muscle, pancreas, prostate, blood cells (including lymphocytes and other immune system cells), and brain. Specific cancers contemplated for treatment include carcinomas, Karposi's sarcoma, melanoma, mesothelioma, soft tissue sarcoma, pancreatic cancer, lung cancer, leukemia (acute lymphoblastic, acute myeloid, chronic lymphocytic, chronic myeloid, and other), and lymphoma (Hodgkin's and non-Hodgkin's), and multiple myeloma. Other examples of cancers that can be treated according to the methods disclosed herein are adrenocortical carcinoma, adrenocortical carcinoma, cerebellar astrocytoma, basal cell carcinoma, bile duct cancer, bladder cancer, bone cancer, brain tumor, breast cancer, Burkitt's lymphoma, carcinoid tumor, central nervous system lymphoma, cervical cancer, chronic myeloproliferative disorders, colon cancer, cutaneous T-cell lymphoma, endometrial cancer, ependymoma, esophageal cancer, gallbladder cancer, gastric (stomach) cancer, gastrointestinal carcinoid tumor, germ cell tumor, glioma, hairy cell leukemia, head and neck cancer, hepatocellular (liver) cancer, hypopharyngeal cancer, hypothalamic and visual pathway glioma, intraocular melanoma, retinoblastoma, islet cell carcinoma (endocrine pancreas), laryngeal cancer, lip and oral cavity cancer, liver cancer, medulloblastoma, Merkel cell carcinoma, squamous neck cancer with occult mycosis fungoides, myelodysplastic syndromes, myelogenous leukemia, nasal cavity and paranasal sinus cancer, nasopharyngeal cancer, neuroblastoma, non-small cell lung cancer, oral cancer, oropharyngeal cancer, osteosarcoma, ovarian cancer, pancreatic cancer, paranasal sinus and nasal cavity cancer, parathyroid cancer, penile cancer, pheochromocytoma, pineoblastoma and supratentorial primitive neuroectodermal tumor, pituitary tumor, plasma cell neoplasm/multiple myeloma, pleuropulmonary blastoma, prostate cancer, rectal cancer, renal cell (kidney) cancer, retinoblastoma, rhabdomyosarcoma, salivary gland cancer, Ewing's sarcoma, soft tissue sarcoma, Sezary syndrome, skin cancer, small cell lung cancer, small intestine cancer, supratentorial primitive neuroectodermal tumors, testicular cancer, thymic carcinoma, thymoma, thyroid cancer, transitional cell cancer of the renal pelvis and ureter, trophoblastic tumor, urethral cancer, uterine cancer, vaginal cancer, vulvar cancer, Waldenström's macroglobulinemia, and Wilms' tumor.

### Administration

The disclosed compounds can be administered either sequentially or simultaneously in separate or combined pharmaceutical formulations. When one or more of the disclosed compounds is used in combination with a second therapeutic agent the dose of each compound can be either the same as or differ from that when the compound is used alone. Appropriate doses will be readily appreciated by those skilled in the art.

The term “administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents.

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration, or at continuous or distinct intervals as can be readily determined by a person skilled in the art.

The compounds disclosed herein, and compositions comprising them, can also be administered utilizing liposome technology, slow release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 99%, and especially, 1 and 15% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell comprises attaching the compounds to a protein or nucleic acid that is targeted for delivery to the target cell. U.S. Pat. No. 6,960,648 and U.S. Application Publication Nos. 20030032594 and 20020120100 disclose amino acid sequences that can be coupled to another composition and that allows the composition to be translocated across biological membranes. U.S. Application Publiation No. 20020035243 also describes compositions for transporting biological moieties across cell membranes for intracellular delivery. Compounds can also be incorporated into polymers, examples of which include poly (D-L lactide-co-glycolide) polymer for intracranial tumors; poly[bis (p-carboxyphenoxy)propane:sebacic acid] in a 20:80 molar ratio (as used in GLIADEL); chondroitin; chitin; and chitosan.

Therapeutic application of compounds and/or compositions containing them can be accomplished by any suitable therapeutic method and technique presently or prospectively known to those skilled in the art. Further, compounds and compositions disclosed herein have use as starting materials or intermediates for the preparation of other useful compounds and compositions.

Compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts, hydrates, or analogs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, compounds and agents disclosed herein can be applied in as a liquid or solid. However, it will generally be desirable to administer them topically to the skin as compositions, in combination with a dermatologically acceptable carrier, which can be a solid or a liquid. Compounds and agents and compositions disclosed herein can be applied topically to a subject's skin to reduce the size (and can include complete removal) of malignant or benign growths, or to treat an infection site. Compounds and agents disclosed herein can be applied directly to the growth or infection site. Preferably, the compounds and agents are applied to the growth or infection site in a formulation such as an ointment, cream, lotion, solution, tincture, or the like. Drug delivery systems for delivery of pharmacological substances to dermal lesions can also be used, such as that described in U.S. Pat. No. 5,167,649.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user. Examples of useful dermatological compositions which can be used to deliver a compound to the skin are disclosed in U.S. Pat. Nos. 4,608,392; 4,992,478; 4,559,157; and 4,820,508.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art; for example, see U.S. Pat. No. 4,938,949.

Also disclosed are pharmaceutical compositions that comprise a compound disclosed herein in combination with a pharmaceutically acceptable carrier. Pharmaceutical compositions adapted for oral, topical or parenteral administration, comprising an amount of a compound constitute a preferred aspect. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

For the treatment of oncological disorders, compounds and agents and compositions disclosed herein can be administered to a patient in need of treatment prior to, subsequent to, or in combination with other antitumor or anticancer agents or substances (e.g., chemotherapeutic agents, immunotherapeutic agents, radiotherapeutic agents, cytotoxic agents, etc.) and/or with radiation therapy and/or with surgical treatment to remove a tumor. For example, compounds and agents and compositions disclosed herein can be used in methods of treating cancer wherein the patient is to be treated or is or has been treated with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively. These other substances or radiation treatments can be given at the same as or at different times from the compounds disclosed herein.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1: α₁-Adrenoceptor Ligands Inhibit Chemokine Receptor Heteromerization Partners of α1_{B/D}-Adrenoceptors via Interference with Heteromer Formation

This example demonstrates that agonist and antagonist binding to α_{1B/D}-AR reduces the heteromerization affinity of the receptor partners, leading to depletion of α_{1B/D}-AR:CR heteromers on the cell surface, inhibition of CR-mediated Gαi1 activation and signaling in vitro, and inhibition of CR-mediated leukocyte infiltration in a murine air pouch model in vivo. Agonists and antagonists at one GPCR can act as functional antagonists at heteromerization partners of their target receptors.

### Materials and Methods

### Proteins, Antibodies and Reagents

Chemokine (C-C motif) ligand (CCL) 1 (CCL1), CCL2 and chemokine (C-X-C motif) ligand 12 (CXCL12) were purchased from Protein Foundry (Milwaukee, WI). Antibodies were obtained from Abcam (Cambridge, United Kingdom): anti-α_{1B}-AR (host: rabbit; catalog: ab169523), anti-α_{1D}-AR (host: rabbit; catalog: ab84402), anti-CXCR4 (host: goat, ab1670), anti-HA (host: rabbit; catalog: ab9110); LifeSpan Biosciences (Seattle, WA): anti-CCR8 (host: goat; catalog: LS-C187704); R&D Systems (Minneapolis, MN): anti-CCR1(host: mouse; catalog: MAB145), anti-CCR2 (host: mouse; catalog: MAB48607), allophycocyanin (APC) conjugated anti-mouse CD45 (host: rat; catalog: FAB114A), APC conjugated Immunoglobulin G 2B (IgG2B) isotype control (host: rat; catalog: ICO13A), Immunoglobulin G (IgG) isotype control (host: rabbit; catalog: MAB150), IgG isotype control (host: mouse; catalog: MAB004), and IgG isotype control (host: goat; catalog: AB-108-C); and Sigma-Aldrich (St. Louis, MO): anti-FLAG (host: mouse; catalog: F3165).

Phenylephrine (PE), phentolamine (PT), norepinephrine (NE), lipopolysaccharide (LPS) from Pseudomonas aeruginosa (serotype 10.22; source strain ATCC 27316) and all proximity ligation assay (PLA) reagents were purchased from Sigma-Aldrich.

### Cells

THP-1 cells, THP-1_ADRA1B^{KO} cells, HEK293T cells and HTLA cells were as described and cultured as reported previously (Gao, 2022; Gao, 2021; Gao, 2020; Enten, 2022).

### Plasmids

α_{1b}-AR cDNA was from FLAG-α_{1b}-AR-Tango (#66214, Addgene) deposited by the laboratory of Dr. Bryan Roth. Human and mouse CCR2 cDNA was synthesized by Twist Bioscience, as described (Gao, 2022). FLAG-CCR2-Tango was generated by insertion of CCR2 cDNA in the empty TANGO vector. α_{1b}-AR and CCR2 cDNA were fused at Age I and Xba I sites with either RlucII or EYFP at C-terminus to form α_{1b}-AR-RlucII and CCR2-EYFP, as described (Gao, 2022). Gαi1-Rluc8, Gβ3 and Gγ9-GFP2 were from Addgene deposited by the laboratory of Dr. Bryan Roth (Olsen, 2020).

### Saturation Bioluminescence Resonance Energy Transfer (BRET) to Assess Receptor Heterodimerization

BRET assays were performed as described previously (Gao, 2022; Gao, 2021; Gao, 2020; Enten; 2022; Albee, 2018; Gao, 2020). In brief, HEK293T cells were seeded in 24-well plates and transfected using TransIT-2020 Transfection Reagent (Mirus Bio, Madison, WI). For saturation BRET assays, α_{1b}-AR-RlucII at a fixed amount of 6 ng was transfected alone or with increasing amounts of CCR2-EYFP. For each transfection, empty vector pcDNA3.1 was added to maintain the total DNA amount constant. After an overnight incubation, cells were seeded in poly-L-lysine coated 96-well white plates and incubated again overnight. Cells were then washed with PBS and fluorescence was measured in a Biotek Synergy HT4 plate reader (λexcitation/emission 485/528 nm). For BRET measurements, coelenterazine H was added at a final concentration of 5 μM. After 3 min incubation at room temperature, cells were treated with vehicle, 10 μM PE or PT for 5 min at room temperature. Luminescence was measured at 485 nm and 528 nm. The BRET signal was calculated as the ratio of the relative luminescence units (RLU) measured at 528 nm over RLU at 485 nm. The net BRET is calculated by subtracting the BRET signal of the wells transfected with α_{1b}-AR-RLucII alone. The net BRET ratios are expressed as a function of fluorescence/total luminescence.

### Gαi Activation Assay

HEK293T cells were plated in a 6-well plate and transfected with 0.2 μg each of Gαi1-Rluc8, Gβ3 and Gγ9-GFP2 (Trupath) together with 0.2 μg of mouse or human CCR2 with either pcDNA3.1 or α_{1b}-AR. One day after transfection, cells were trypsinized and replated to 96-well poly-L lysine precoated plates. After overnight incubation, cells were replaced with 0.1% glucose/PBS. Prolume Purple (NanoLight Tech.) or coelenterazine 400a in a final concentration of 5 μM were added in experiments with human or mouse CCR2, respectively, and incubated at room temperature for 3 min. Ligands at various concentrations were added to cells and incubated at room temperature for 5 min before luminescence was measured at 410 nm and 515 nm. The BRET signal was calculated as the ratio of the relative luminescence units (RLUs) measured at 515 nm over RLUs measured at 410 nm. The BRET changes were calculated by subtracting the BRET signal of untreated cells.

### PRESTO-Tango β-Arrestin Recruitment Assay

The assays were performed as described previously (Albee, 2017; Gao, 2022; Albee, 2018; Eby, 2017; Cheng, 2017). HTLA cells were seeded in a 6-well plate and transfected with 750 ng FLAG-CCR2-Tango together with 750 ng pcDNA3.1 or HA-α_{1b}-AR using TransIT-2020 Transfection Reagent. The next day, cells (75,000 cells/well) were plated onto poly-L-Lysine pre-coated 96-well plates and allowed to attach to the plate surface for at least 4 hours prior to treatment. Cells were treated with receptor ligands at the concentrations indicated in the figure legends for 2 h and then replaced with fresh full medium and incubated overnight at 37° C., 5% CO₂ in a humidified environment. The following morning, medium was replaced with a 100 μL 1:10 mixture of Bright-Glo (Promega) and PBS. Plates were then incubated at room temperature for 10 min before measuring luminescence on a Biotek Synergy II plate reader. Relative luminescence units (RLU) are expressed as fold changes of RLU vs unstimulated cells.

### BRET Assay for β-Arrestin Recruitment

β-arrestin recruitment was measured by BRET, as described (Armando, 2014; Berchiche, 2011). In brief, HEK293T cells were plated in a 6-well plate and transfected with 0.1 μg of CCR2-RluII and 1.9 μg β-arrestin-YFP with 0.1 μg pcDNA3.1 or α_{1b}-AR. One day after transfection, cells were re-plated into 96-well poly-L lysine precoated plates. After overnight incubation, cells were replaced with 0.1% glucose/PBS. Coelenterazine H in a final concentration of 5 μM was added to cells and incubated at room temperature for 3 min. Ligands at various concentrations were added to the cells and incubated at room temperature for 10 min before luminescence was measured at 485 nm and 528 nm. The BRET signal was calculated as the ratio of the relative luminescence units (RLUs) measured at 528 nm over RLUs measured at 485 nm. The BRET changes were calculated by subtracting the BRET signal of untreated cells.

### CCR2 Internalization

To examine receptor internalization in THP-1 cells, cells were incubated with 100 nM CCL2 or with 100 nM CCL2 plus 1 μM PE for 0-45 min at 37° C. Cells were then cooled on ice and washed with 50 mM glycine buffer, pH 2.7, 150 mM NaCl, followed by washing with ice-cold PBS. Cell surface CCR2 was probed with anti-CCR2 and corresponding secondary Alexa 488-conjugated antibodies. Receptor cell surface expression levels are examined by flow cytometry and expressed as the median fluorescence intensity (MFI) in percent of the MFI measured at 0 min (=100%).

### Gene Silencing by RNA Interference

Gene silencing was performed with siRNAs from Horizon Discovery Biosciences (Cambridge, UK). In brief, THP-1 cells were incubated for 3 days in Accell transfection media with α_{1B}-AR-targeting or non-targeting (NT) siRNA at a concentration of 1 μM. Cells were then replaced with culture medium RPMI 1640. Cells were utilized on day 4 for experimentation.

### Cyclic AMP Measurement

Cyclic AMP amounts were measured using the cAMP complete enzyme immunoassay kit, acetylated format (Enzo Life Sciences). 4×10⁵ cells were incubated with forskolin/isobutylmethylxanthine (10 μM/0.5 mM final concentrations) in 0.1% BSA/10 mM Hepes-buffered RPMI and treated with CCL2 in the presence or absence of 1 μM PE, 1 μM PT or 100 nM NE at 37° C. for 15 min. The cells were lysed by incubating with 0.1 M HCl at room temperature for 20 min. The cAMP amounts in the lysate supernatant were measured following the manufacturer's instructions.

### Proximity Ligation Assay (PLA)

PLAs were performed as described in detail previously (Tripathi, 20415; Evans, 2016; Albee, 2017; Albee, 2021; Enten, 2022; Albee, 2018; Gao, 2020). THP-1 cells were incubated (30 min at 37° C. in a humidifying chamber, 5% CO₂) in RPMI 1640 supplemented with 10% FBS and 10 μM of either phenylephrine or phentolamine. Monolayer cell deposits were then prepared on glass slides (Thermo Fisher Scientific, Waltham, MA) by centrifugation at 800×g with a Cytospin 4 centrifuge (Thermo Fisher Scientific) and isolated into individual wells using a water-repellent solution (super PAP pen, Thermo Fisher Scientific). All cells were fixed with 4% (wt/vol) paraformaldehyde (15 min at room temperature), washed with PBS, and then blocked overnight at 4° C. with Duolink™ PLA blocking reagent (Sigma-Aldrich). Blocked slides were incubated (105 min at 37° C. in a humidifying chamber) with indicated primary antibody(s) in dilutions of 1 μg/mL corresponding to the receptor(s) of interest. IgG isotype antibodies were utilized as a control. Cells were then washed with PBS and incubated (60 min at 37° C. in a humidifying chamber) with secondary species-specific antibodies conjugated to plus and minus PLA probes (1:5 Duolink™ PLA dilution Buffer) (Sigma-Aldrich). Cells were washed with Duolink™ PLA wash buffer A (Sigma-Aldrich) and then incubated with Duolink™ PLA probe ligation reagent (Sigma-Aldrich) (30 min at 37° C. in a humidifying chamber). Subsequently, cells were washed again in wash buffer A and then incubated with PLA amplification reagent (Sigma-Aldrich) (105 min at 37° C. in a humidifying chamber). After amplification, cells were washed twice with Duolink™ PLA wash buffer B (Sigma-Aldrich) and then once with a 0.01× dilution of PLA wash buffer B in ddH2O. Treated slides were dried and mounted with 50 μL per well of Duolink™ in situ mounting medium with DAPI (Sigma-Aldrich) overnight at −20° C. PLA signals (Duolink™ in situ detection reagents red; λexcitation/emission 598/634 nm) were identified as red fluorescent spots under a Keyence (Osaka, Japan) BZ-X710 fluorescence microscope (60×/1.50 oil) at room temperature. PLA signals were quantified using ImageJ (NIH). Images were imported in merged TIFFs containing both signal and nuclei channels. Merged images were visually verified for analytical quality. Comparisons and statistical analyses were performed only when PLA assays were performed on the same day in parallel experiments. Fluorescence microscopy was performed with identical settings. For each experiment and condition, ten randomly selected nonoverlapping vision fields were analyzed.

### Murine Air Pouch Model

All procedures were performed according to the National Institutes of Health Guidelines for Use of Laboratory Animals and were approved by the Institutional Animal Care and Use Committee of the University of South Florida (IS00010570). Male and female C57BL/6 mice (20-25 g) were obtained from Envigo (Indianapolis, IN). Six days prior to the start of the experiments, mice were anesthetized with 1.7% sevoflurane, the dorsal cervical/thoracic region was shaved, disinfected with 70% ethanol and 3 mL of sterile air were injected subcutaneously (s.c.) to create an air pouch. On the third day, mice were re-anesthetized, their dorsal cervical/thoracic region were re-sterilized, and an additional 3 mL of sterile room air were injected into the existing air pouch. On the sixth day, mice were re-anesthetized and vehicle (2.5 mL normal saline), 2.5 mg/kg LPS, 6 μg CCL2 or 2 μg CXCL12 plus vehicle, phenylephrine (100 mg/kg, 50 mg/kg, 25 mg/kg, 10 mg/kg) or phentolamine (50 mg/kg, 25 mg/kg, 10 mg/kg, 5 mg/kg) in a total volume of 2.5 mL normal saline were injected into the air pouch. 24 h later, mice were euthanized by CO₂ inhalation, and air pouch cells harvested by lavage with 5 mL of lavage fluid (PBS, 5 mM ethylenediaminetetraacetic acid [EDTA], 3% FBS). 5 ml ice cold lavage fluid was injected into each air pouch using a syringe with a 23-G, 1-inch needle, the air pouch was gently massaged for 60 seconds, the lavage fluid was harvested using the same syringe and needle and the recovered volume was recorded. On average, 75% of the lavage fluid could be recovered. The harvested lavage fluids were then passed through a Falcon™ 40 μm nylon mesh filter (Corning, Corning, NY) and centrifuged (500× g for 10 min, 4° C.). Erythrocytes in the cell pellets were lysed with Invitrogen 1× RBC lysis buffer and the harvested air pouch cells analyzed by flow cytometry.

### Flow Cytometry

Flow cytometry was used to evaluate receptor expression, as described (Tripathi, 2015; Gao, 2022; Gao, 2020), and for the analyses of harvested air pouch cells. For the analysis of receptor expression, cells were incubated with anti-HA, anti-FLAG, anti-α_{1B}-AR, anti-CCR2 or anti-CCR8 (1:100 dilution), followed by incubation with corresponding secondary antibodies conjugated with Alexa Fluor 488, Alexa Fluor 594 or Alexa Fluor 647 (Thermofisher Scientific, 1:100 dilution). The fluorescence intensities of at least 10,000 cells were recorded and analyzed using the FlowJo software (Tree Star, Ashland, OR).

Harvested air pouch cells were incubated with APC-conjugated anti-mouse CD45 or APC-conjugated immunoglobulin G 2B (IgG2B; isotype control) for 145 min in the dark at room temperature. Cells were then washed with 1 mL PBS and fixed in 0.5 mL 1% paraformaldehyde. Cells were then washed again, resuspended in PBS, and the cell-associated light scatter and fluorescence were determined with a LSRII instrument (Becton Dickinson). Sample cellularity/mL was calculated by dividing the total number of cells counted per sample by the volume of recovered air pouch lavage fluid.

### Data Analyses and Statistics

Data are presented as mean±standard error (SE). Data were analyzed by Student's t-test, 1-way analysis of variance (ANOVA) or two-way ANOVA with Bonferroni's multiple comparisons tests, as appropriate. Titration curves were analyzed with nonlinear regression analyses. Best-fit values were compared with the extra-sum-of-squares F test. All data analyses were calculated with the GraphPad Prism program (GraphPad Software Version 9.4.1, Aug. 8, 2022). A two-tailed p<0.05 was considered significant.

### Results and Discussion

α_{1b}-AR: CCR2 Heteromerization Facilitates CCR2-Mediated Gαi1 Activation

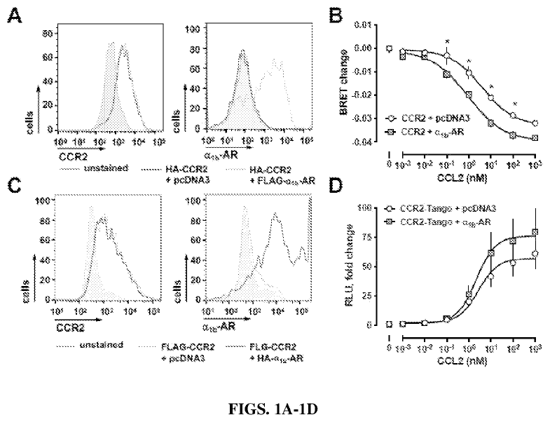

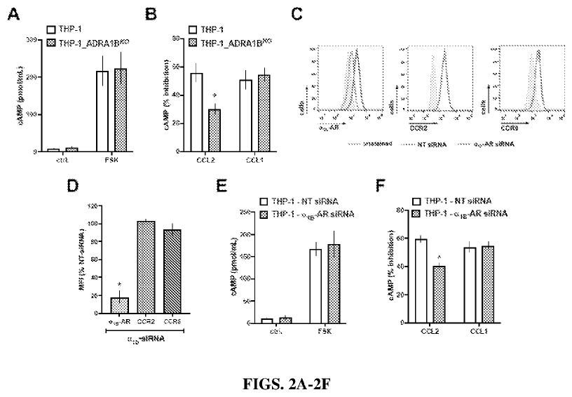

To understand how α_{1B/D}-ARs regulate signaling of their CR heteromerization partners, α_{1b}-AR and chemokine (C-C motif) receptor 2 (CCR2) were selected as prototypical heteromerization partners to determine whether α_{1B}-AR modulates agonist-induced coupling of CCR2 to its signaling transducers Gαi and β-arrestin. TRUPATH bioluminescence resonance energy transfer (BRET) biosensors were employed to measure Gαi1 activation, and the PRESTO-Tango cell system to measure β-arrestin recruitment upon activation of CCR2 with chemokine (C-C motif) ligand 2 (CCL2) in cells that were transfected with CCR2 plus pcDNA3 or with CCR2 plus α_{1b}-AR (Olsen, 2020; Kroeze, 2015). Flow cytometry confirmed that CCR2 expression was comparable, and that α_{1b}-AR was expressed under our experimental conditions (FIGS. 1A, 1C). As shown in FIG. 1B, in cells transfected with CCR2 alone, CCL2 dose-dependently induced dissociation of the heterotrimeric Gαiβγ complex. The determined EC₅₀ of CCL2 was 3.31±nM and bottom plateau was −0.03±0.001. In cells transfected with CCR2 plus α_{1b}-AR, potency and efficacy of CCL2 to activate Gαi1 were significantly increased (EC₅₀: 0.94±0.2 nM, p<0.01 vs. CCR2 alone; bottom plateau: −0.037±0.001, p<0.01 vs. CCR2 alone). The presence of α_{1b}-AR, however, did not significantly affect β-arrestin recruitment to CCR2 upon stimulation with CCL2 (FIG. 1D). To confirm that the presence of α_{1b}-AR facilitates CCR2 mediated activation of Gαi and to test whether our findings in a recombinant system can be translated to endogenously expressed receptors, cAMP concentrations were measured as a proximal signaling read-out of CR-mediated Gαi activation in wild-type THP1 cells and in CRISPR/Cas 9 gene edited THP-1 cells that lack α_{1B}-AR (THP-1_ADRA1B^{KO}) (FIG. 2A/B). It should be noted that CCL2 is a cognate agonist of CCR2, CCR3 and CCR5, all of which are known to be expressed in THP-1 cells (Martinelli, 2001; Giri, 2005). The binding affinity of CCL2 for CCR2, however, is approximately 100-fold higher than the binding affinity of CCL2 for CCR3 and CCR5 (Napier, 2005; Daugherty, 1996; Coulin, 1997). Furthermore, it was shown previously that CCR2 is the key driver of CCL2-induced chemotaxis in THP-1 cells and that CCR3 and CCR5 are also heteromerization partners of α₁-ARs (Enten, 2022). As shown in FIG. 2A, unstimulated and forskolin-stimulated cAMP concentrations were indistinguishable in THP-1 and THP-1_ADRA1B^{KO} cells. While CCL2 reduced cAMP levels in forskolin-stimulated THP-1 cells by 56±6%, the efficacy of CCL2 to inhibit cAMP production was significantly reduced to 30±4% of cells not exposed to CCL2 in forskolin-stimulated THP-1_ADRA1B^{KO} cells (p<0.05 vs. THP-1 cells, FIG. 2B). When cells were exposed to CCL1, which is a selective agonist of CCR8, a CR that does not heteromerize with α_{1B/D}-AR in THP-1 cells (Enten, 2022), the inhibition of forskolin-stimulated cAMP production was indistinguishable in THP1 and THP-1_ADRA1B^{KO} cells (FIG. 2B). To corroborate the findings in THP-1_ADRA1B^{KO} cells and to exclude that such effects are clonal specific (Luttrell, 2018), cAMP concentrations were then measured in THP-1 cells incubated with non-targeting siRNA and α_{1B}-AR-targeting siRNA (FIG. 2C-F). Quantification of receptor expression levels by flow cytometry showed that incubation of THP-1 cells with α_{1B}-AR-targeting siRNA reduced α_{1B}-AR expression by 82±7% and did not affect expression of CCR2 or CCR8, when compared with THP-1 cells incubated with non-targeting siRNA (FIG. 2C/D). While siRNA knockdown of α_{1B}-AR did not affect forskolin-stimulated cAMP concentrations (FIG. 2E) or the inhibition of forskolin-stimulated cAMP production after incubation with CCL1, α_{1B}-AR knockdown significantly reduced the efficacy of CCL2 to inhibit cAMP production (% inhibition: non-targeting siRNA—66±5%; α_{1B}-AR siRNA—46±4%, p<0.05) (FIG. 2F). The observation that the reduction of the efficacy of CCL2 to inhibit forskolin stimulated cAMP production was more pronounced in THP-1_ADRA1B^{KO} cells than in THP-1 cells after incubation with α_{1B}-AR siRNA is consistent with incomplete siRNA silencing of α_{1B}-AR. Thus, the findings from CRISPR/Cas 9 gene edited THP-1 cells and from THP-1 cells after siRNA knockdown of α_{1B}-AR indicate that α_{1B}-AR per se enhances agonist-induced coupling of CCR2 to Gαi and support the notion that the observed effects of α_{1B}-AR on CR signaling are specific for CR heteromerization partners of α_{1B}-AR (Enten, 2022).

### Agonist and Antagonist Binding to α_{1B}-AR Reduce the Heteromerization Affinity Between α_{1b}-AR and CCR2 and Inhibit Gαi1 Activation via CCR2

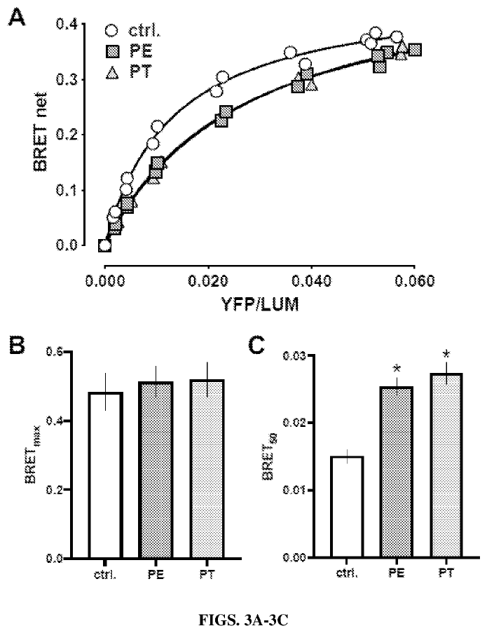

Because observations imply that interference with α_{1b}-AR:CCR2 heteromerization impairs CCR2 mediated activation of Gαi1, it was tested whether ligand binding to α_{1b}-AR affects the ability of α_{1b}-AR to heteromerize with CCR2 in saturation BRET experiments. FIG. 3A shows a typical saturation BRET experiment in HEK293T cells that were transfected with a constant amount of α_{1b}-AR-RLucII and increasing amounts of CCR2-EYFP, and FIG. 3B/C the BRETₘₐₓ and BRET₅₀ values from 4 independent experiments. Consistent with our previous observations, the BRET net signal showed hyperbolic progression with increasing energy acceptor: donor ratios, which is in agreement with constitutive heteromerization between CCR2 and α_{1b}-AR (Gao, 2022; Enten, 2022). While phenylephrine and phentolamine did not affect BRETₘₐₓ (FIG. 3B), both ligands significantly increased BRET₅₀ values (FIG. 3C), indicating that both agonist and antagonist binding to α_{1b}-AR reduces the heteromerization affinity between α_{1b}-AR and CCR2.

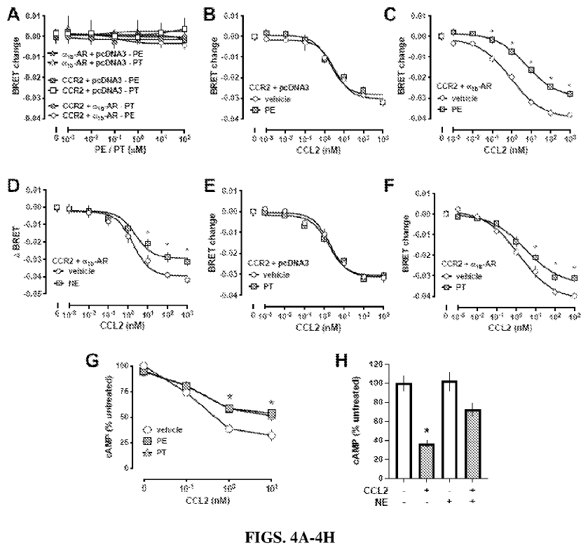

The effects of α_{1b}-AR ligands on CCR2 mediated activation of Gαi1 in BRET experiments with Gαi1βγ biosensors are shown in FIGS. 4A-4F. Phenylephrine and phentolamine did not activate Gαi1 in cells transfected with α_{1b}-AR alone, CCR2 alone or with CCR2 plus α_{1b}-AR (FIG. 4A). While phenylephrine did not affect CCL2-induced Gαi1 activation in cells transfected with CCR2 alone (FIG. 4B), phenylephrine reduced the potency and efficacy CCL2 to induce Gαi activation in cells transfected with CCR2 plus α_{1b}-AR (EC₅₀ of CCL2: vehicle—0.9±0.25 nM, phenylephrine—5.6±2.4 nM, p<0.001; bottom plateau:vehicle—−0.04±0.002, phenylephrine—−0.03±0.002, p<0.05) (FIG. 4C). Similarly, in cells transfected with CCR2 plus α_{1b}-AR, the endogenous α_{1b}-AR agonist norepinephrine reduced the efficacy CCL2 to induce Gαi activation (EC₅₀ of CCL2: vehicle—1.3±0.4 nM, norepinephrine—1.9±0.8 nM, p>0.05; bottom plateau: vehicle—−0.04±0.002, phenylephrine—−0.031±0.002, p<0.05) (FIG. 4D).

As observed with phenylephrine and norepinephrine, phentolamine did not affect CCL2-induced Gαi activation in cells transfected with CCR2 alone (FIG. 4E). In the presence of CCR2 and α_{1b}-AR, phentolamine reduced the efficacy of CCR2-mediated Gαi activation upon CCL2 stimulation without significantly affecting the potency of CCL2 (EC₅₀ of CCL2:vehicle—1.5±0.5 nM, phentolamine—2.7±1.0 nM, p>0.05; bottom plateau: vehicle—−0.042±0.002, phentolamine—−0.033±0.002, p<0.05) (FIG. 4F). Consistent with these observations in recombinant systems, phenylephrine and phentolamine (FIG. 4G) as well as the endogenous α_{1b}-AR agonist norepinephrine (FIG. 4H) inhibited the effects of CCL2 on cAMP production in forskolin-stimulated THP-1 cells. In combination with our findings on the effects of ligand-free α_{1B}-AR on CCR2 mediated activation of Gαi1, these observations suggest that ligand binding to α_{1B}-AR interferes with α_{1B}-AR: CCR2 heteromerization, which in turn results in impaired activation of Gαi1 by CCR2. Moreover, our observations on the effects of norepinephrine imply that endogenous catecholamines inhibit the function of the chemokine receptor heteromerization partners of α_{1B}-AR.

### Agonist Binding to α_{1b}-AR Cross-Recruits β-Arrestin to CCR2 and Enhances CCL2-Mediated β-Arrestin Recruitment to CCR2 Within CCR2:α_{1b}-AR Heteromers

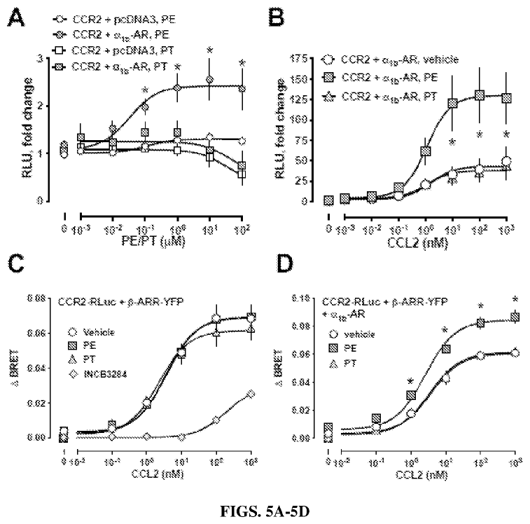

Because it was observed previously that agonist binding to α_{1b}-AR within the α_{1b}-AR:CXCR4 heteromeric complex leads to cross-recruitment of β-arrestin to CXCR4 and that agonist binding to CCR2 within CCR2:α_{1b}-AR heteromers cross-recruits β-arrestin to α_{1b}-AR (Gao, 2018; Gao; 2022), it was tested whether α₁-AR ligands would also affect β-arrestin recruitment to CCR2. It was observed that phenylephrine and phentolamine did not induce β-arrestin recruitment to CCR2-Tango in the absence of α_{1b}-AR (FIG. 5A, open symbols). When cells were co-transfected with CCR2-Tango plus α_{1b}-AR, however, phenylephrine dose-dependently induced β-arrestin recruitment to CCR2-Tango with an EC₅₀ of 33±27 nM, whereas phentolamine had no effect (FIG. 5A, grey symbols). To evaluate how ligand binding to α_{1b}-AR affects agonist-induced β-arrestin recruitment to CCR2, we then determined the dose-response characteristics of CCL2 in the presence and absence of the α_{1b}-AR ligands in cells co-transfected with CCR2-Tango plus α_{1b}-AR (FIG. 5B). CCL2 alone induced β-arrestin recruitment to CCR2 with an EC₅₀ of 1.6±1.3 nM. As compared with the efficacy of phenylephrine to induce β-arrestin recruitment to CCR2 (2.4±0.14 fold change RLU; FIG. 5B), the efficacy of CCL2 to recruit β-arrestin to CCR2 was 18-fold higher (43±5 fold change RLU). In cells co-stimulated with CCL2 and phenylephrine, the efficacy of CCL2 to recruit β-arrestin to CCR2 increased 3-fold (130±12 fold change RLU, p<0.001 vs. CCL2 alone), whereas the potency of CCL2 was unaffected (EC₅₀:1.1±0.7 nM). In contrast, phentolamine did not affect CCL2-induced β-arrestin recruitment to CCR2.

To confirm these findings from PRESTO-Tango β-arrestin recruitment assays, which depend on a tetracyclin transactivator (tTA)-dependent luciferase reporter to generate signals hours after receptor activation, BRET was performed to provide direct biophysical evidence for the association between CCR2 and β-arrestin within minutes after agonist binding. As shown in FIG. 5C, CCL2 increased BRET between CCR2-Rluc and β-arrestin-YFP with an EC₅₀ of 3.5±0.9 nM. The BRET signal was not affected by phenylephrine or phentolamine and could be inhibited with the selective CCR2 inhibitor INCB3284 (Xue, 2011). As observed in PRESTO-Tango assays, phenylephrine increased the efficacy of CCL2 to induce β-arrestin recruitment to CCR2, whereas phentolamine did not affect CCL2 induced BRET changes in cells co-expressing CCR2-Rluc, β-arrestin-YFP and α_{1b}-AR (EC₅₀ of CCL2 (nM): vehicle—3.7±0.5; phenylephrine—2.6±0.5; phentolamine—3.4±0.7 (p>0.05 vs vehicle for both); Top plateau: vehicle—0.06±0.001; phenylephrine—0.084±0.0.002 (p<0.05 vs vehicle); phentolamine—0.061±0.002 (p>0.05 vs vehicle)) (FIG. 5D). Given that β-arrestin recruitment is closely involved in the internalization of agonist-activated CCR2 (Berchiche, 2011), findings suggest that agonist and antagonist binding α_{1B}-AR within α_{1B}-AR: CCR2 heteromers will have differential effects on CCR2 expression in cells.

### α₁-AR Ligands Interfere with Heteromerization Between α_{1B/D}-AR and Their Chemokine Receptor Partners

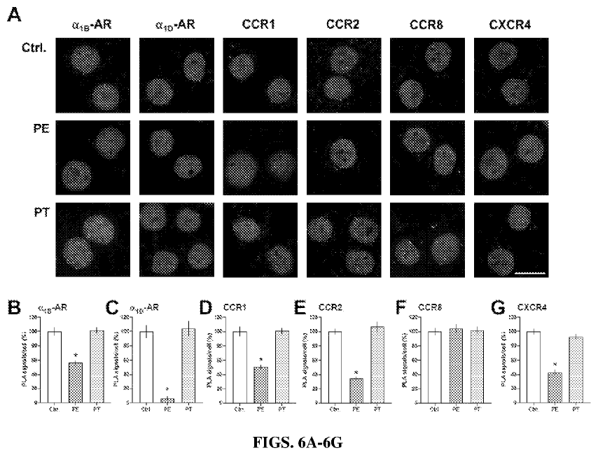

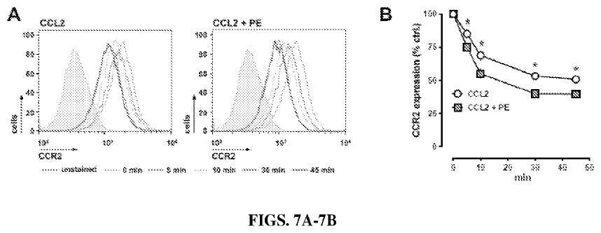

To test whether the observed effects of the α₁-AR ligands on β-arrestin recruitment to CCR2 are associated with corresponding changes of receptor expression levels, and whether effects on CCR2 are generalizable to other CR heteromerization partners of α_{1B/D}-AR, proximity ligation assays (PLA) were employed in THP-1 cells to visualize and quantify individual receptors and receptor-receptor interactions. For these experiments, α_{1B/D}-ARs and the heteromerization partners CCR1, CCR2 and CXCR4 were selected; CCR8 was used as a control CR that does not interact with α1B/D-AR (Enten, 2022). FIG. 6A shows typical PLA images for the detection of individual receptors in cells treated with vehicle (top), phenylephrine (center) and phentolamine (bottom), and FIG. 6B the quantification of PLA signals from three independent experiments. As expected, α1B/D-AR, the heteromerization partners CCR1, CCR2 and CXCR4, as well as non-interaction partner CCR8 could be detected in THP-1 cells by PLA (Enten, 2022). When compared with vehicle treated cells, phenylephrine treatment reduced cell surface expression of α_{1B/D}-AR and of the chemokine receptor heteromerization partners CCR1, CCR2 and CXCR4. The expression of CCR8 was not affected by phenylephrine. Incubation of THP-1 cells with phentolamine did not affect receptor expression levels of individual receptors. These findings agree with previous observations (Gao, 2018; Xiu, 2013) and are consistent with the assumption that agonist binding to α_{1B/D}-AR cross-recruits β-arrestin to CCR2 and to other CR heteromerization partners, leading to co-internalization of the receptor partners. Because CCL2-induced β-arrestin recruitment to CCR2 was significantly enhanced when cells were co-stimulated with phenylephrine (FIG. 3H), we utilized flow cytometry to measure CCR2 cell surface expression in THP-1 cells stimulated with CCL2 alone or with CCL2 plus phenylephrine. Consistent with the effects of phenylephrine on CCL2-induced β-arrestin recruitment to CCR2, co-stimulation of THP-1 cells with CCL2 and phenylephrine significantly reduced CCR2 expression, as compared with cells stimulated with CCL2 alone (FIG. 7A/B).

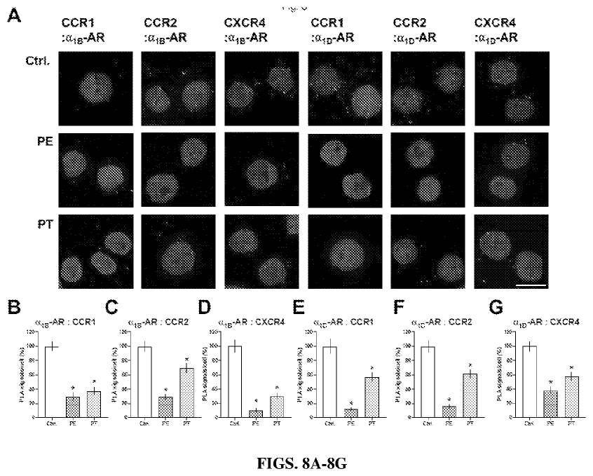

FIG. 8A shows typical images for the detection of receptor-receptor interactions in cells treated with vehicle (top), phenylephrine (center) and phentolamine (bottom), and FIG. 8B the quantification of PLA signals from three independent experiments. Phenylephrine treatment reduced expression levels of heteromeric complexes between α_{1B/D}-ARs and their chemokine heteromerization partners, which agrees with the observed effects of phenylephrine on the expression of the individual receptors. Although phentolamine did not affect expression levels of individual receptors, it also reduced the formation of heteromeric complexes between α_{1B/D}-ARs and their chemokine heteromerization partners. This suggests that the diminished heteromerization affinity of ligand bound α_{1b}-AR that we observed in BRET assays results in reduced formation of α_{1B/D}-AR:CR heteromers on the cell surface.

Agonist-induced β-arrestin recruitment is known to occur secondary to G protein activation (Shenoy, 2011). Because co-stimulation of cells with CCL2 and phenylephrine or norepinephrine inhibited Gαi1 activation via CCR2, these effects cannot be attributed to the enhancing effects of α_{1B}-AR agonists on CCR2-induced β-arrestin recruitment to and subsequent internalization of CCR2, or to β-arrestin mediated signaling events.

Moreover, our previous finding that phenylephrine and phentolamine did not affect the binding affinity of CCR2 for CCL2 (Enten, 2022) suggests that a reduced binding affinity of CCR2 for its agonist is also unlikely to account for the observed inhibition. Therefore, our findings imply that the reduced heteromerization affinities of agonist and antagonist-bound α_{1b}-AR that we observed in BRET experiments impair the formation of heteromeric complexes between α_{1B/D}-ARs and their CR heteromerization partners, thus leading to reduced Gαi activation of the agonist bound CR partners.

### α₁-AR Ligands Prevent LPS, CCL2 and CXCL12-Mediated Air Pouch Infiltration of Leukocytes

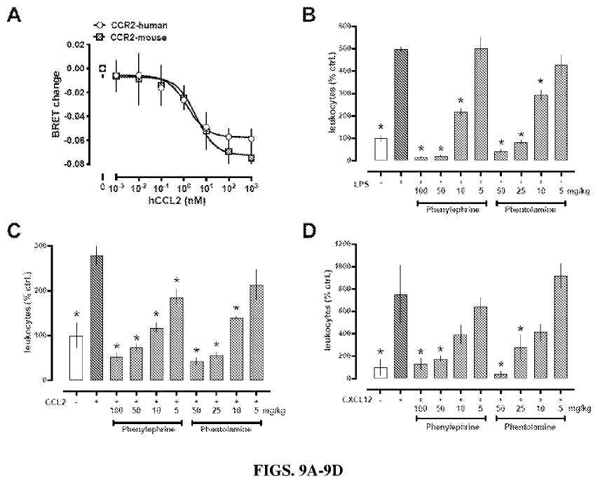

To test whether α₁-AR ligands inhibit chemotaxis of leukocytes mediated via chemokine receptor heteromerization partners in vivo, an air pouch model was employed in C57BL/J6 mice. LPS, CCL2 and CXCL12 were selected as chemotactic agents. While CCL2 and CXCL12 are cognate agonists of the α₁-AR heteromerization partners CCR2, CCR3, CCR5, CXCR4 and ACKR3, LPS induces release of numerous chemokines that mediate chemotaxis via chemokine receptor heteromerization partners of α_{1B/D}-AR (Enten, 2022; Mahalingham, 1999). In these experiments, human CCL2 and CXCL12 were utilized. CXCR4 and CXCL12 are highly conserved among species, show >90% homology between human and mouse proteins, and human and mouse CXCL12 are known to possess interspecies activity (Costs, 2018). Because mouse and human CCR2 and CCL2 share only 66% and 54% sequence identity (UniProt, 2021), respectively, human CCL2-induced Gαi1 activation by human and mouse CCR2 were compared in BRET experiments with Gαi1βγ biosensors. As shown in FIG. 9A, potency and efficacy of human CCL2 to induce dissociation of the heterotrimeric G proteins was indistinguishable in cells transfected with human and mouse CCR2 (human CCR2: EC₅₀—1.4±0.8 nM, bottom plateau—−0.058±0.004; mouse CCR2: EC₅₀—3.5±3.1 nM, bottom plateau—−0.073±0.009; p>0.05 for all). Consistent with their interspecies biological activity, injection of the chemotactic agents into the air pouch resulted in robust cell infiltration at 24 hours post injection (FIG. 9B-D), of which 93±2%, 82±10% and 83±10% expressed leukocyte common antigen (CD45) after LPS, CCL2 and CXCL12 injection, respectively. Based on the analyses of the cell-associated light scatter of CD45 positive cells, 82±4% could be identified as granulocytes and 8±2% as mononuclear cells after LPS injection. After CCL2 and CXCL12 injection, 21±6% and 19±7%, respectively, could be identified as granulocytes, and 48±3% and 37±3%, respectively, as mononuclear cells. When the chemotactic agents were co-injected with increasing doses of phenylephrine or phentolamine, leukocyte infiltration could be inhibited in a dose-dependent manner. Phenylephrine and phentolamine did not affect the relative distribution of mononuclear cells and granulocytes under each experimental condition (p>0.05 vs. chemotactic agent alone). Adverse effects of the α₁-AR ligands were not observed. These findings indicate that the previously described effects of phenylephrine and phentolamine in transwell migration assays with THP-1 cells, human monocytes and human vascular smooth muscle cells in vitro (Gao, 2018; Enten, 2022) can be reproduced in vivo and are also applicable to leukocytes other than monocytes. It should be noted, however, that in the present study phenylephrine and phentolamine prevented leukocyte infiltration in vivo, whereas both drugs did not fully inhibit CCL2 and CXCL12-mediated chemotaxis of THP-1 cells and human monocytes in vitro. It cannot be excluded that the previously observed in vitro effects of the α₁-AR ligands on chemotaxis underestimate in vivo effects, or that species specific differences account for the higher efficacy of the α₁-AR ligands to inhibit leukocyte chemotaxis in the mouse model, it appears also possible that phenylephrine and phentolamine have more pronounced effects on chemotaxis of leukocyte subsets other than monocytes.

### CONCLUSION

In conclusion, heteromerization between α_{1B}-AR and CCR2 facilitates activation of Gαi by CCR2. Agonist and antagonist binding to α_{1B/D}-AR reduces the heteromerization affinity of α_{1B/D}-AR for their CR partners, which reduces the proportion of CRs that exists within CR:α_{1B}-AR heteromers and results in impaired CR signaling and function. These findings provide a molecular basis underlying the regulation of CR function by ligand-free and ligand-bound α_{1B/D}-AR within CR:α_{1B/D}-AR heteromers. It appears likely that conformational re-arrangements of α_{1B/D}-ARs upon ligand binding impair their propensity to heteromerize, which allosterically impairs coupling of the CR partners to Gαi. Moreover, an α₁-AR agonist as well as an α₁-AR antagonist inhibit leukocyte infiltration in response to agonists of chemokine receptor heteromerization partners of α_{1B/D}-AR in vivo. This points toward α_{1B/D}-AR:CR heteromerization as a physiologically relevant mechanism by which the sympathetic nervous system regulates leukocyte positioning and trafficking during the physiological stress or fight-or-flight response, and further suggests that drugs targeting α_{1B/D}-AR affect immune cell trafficking. In combination with our previous finding that CCR2 within CCR2: α_{1B}-AR heteromers regulates the function of recombinant α_{1b}-AR and of α_{1B}-AR in vascular smooth muscle very similar to the effects of α_{1B}-AR on CCR2 that were detected in the present study (Gao, 2022), findings point toward heteromerization between α_{1B}-AR and its chemokine receptor partners as a molecular mechanism by which inflammation, i.e. chemokine release, and physiological stress responses, e.g. catecholamine release, interact with each other to regulate organ and cell function. Moreover, our findings demonstrate that a bona fide agonist and antagonist of one GPCR can act as functional inhibitors of the heteromerization partners of their target receptors on the molecular level and in vivo, which suggests new aspects of the pharmacology and potential side-effect profiles of

GPCR targeting drugs.

### Example 2: Inhibition or Modulation of Inflammation and Cell or Cancel Cell Trafficking with Drugs that Interfere with the Heteromerization of CRs with α₁-AR and AVPR1A

The 7-transmembrane domain (7TM) protein family of chemokine receptors (CRs) is composed of 18 G protein-coupled receptors (GPCRs), 4 atypical chemokine receptors (ACKR1-4) and chemokine (CC motif) receptor-like (CCRL)2/designated ACKR5 pending confirmation. CRs are essential for the regulation of leukocyte positioning, trafficking and recruitment, and play roles in all aspects of inflammation, including numerous disease processes, as diverse as infections, autoimmune diseases, cancer, or tissue injury and repair. Accordingly, CRs are attractive drug targets and the CR antagonists Maraviroc (CCR5 antagonist) and AMD3100 (CXCR4 antagonist) are already FDA approved. Most members of the human CR family form heteromeric complexes with one or more members of the α1-adrenergic receptor (AR) family and with arginine vasopressin receptor 1A (AVPR1A) in recombinant systems. Such heteromeric complexes are detectable in human monocytes and in the monocytic leukemia cell line THP-1. Agonist and antagonist binding to α1-AR and AVPR1A modulate the function of the CRs, and that removal of α1-AR from the cell surface inhibits CR function, e.g., induction of chemotaxis in leukocytes and THP-1 cells, by 82-95%. These findings indicate that CR:α1-AR and CR-AVPR1A heteromers control the function of the CR heteromerization partners, provide a mechanism underlying neuroendocrine control of leukocyte trafficking and offer new opportunities to modulate leukocyte and/or cancer cell trafficking in disease processes.

This example demonstrates both that drugs that interfere with the heteromerization of CRs with α1-AR and AVPR1A can be used to inhibit or modulate inflammation and cell or cancer cell trafficking in numerous disease processes, including but not limited to infections, trauma, autoimmune diseases, cardiovascular diseases or cancer; and that drugs that bind to α1-AR and AVPR1A (agonists and antagonists) can be used to inhibit or modulate inflammation and cell or cancer cell trafficking in numerous disease processes, including but not limited to infections, trauma, autoimmune diseases, cardiovascular diseases or cancer. Such drugs include multiple α1-AR and AVPR1A-targeting drugs that are already FDA approved for other indications.

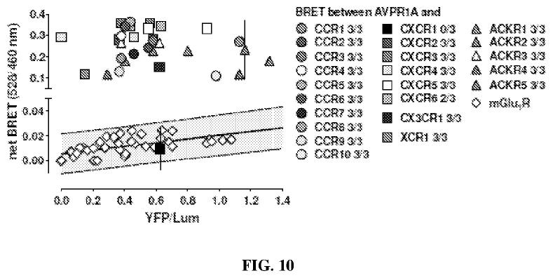

Positive BRET signals were observed for interactions between 21 CRs with AVPR1A in all screening experiments (FIG. 10)

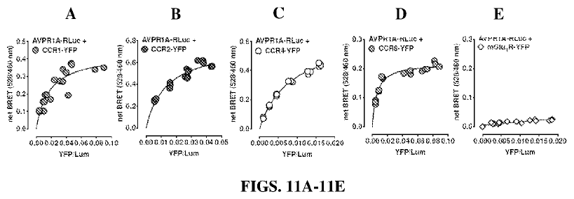

To confirm these findings, saturation BRET experiments were performed and observed hyperbolic progression of the BRET signals for randomly selected CRs that showed positive signals in the BRET screening experiments (FIGS. 11A-11E). These findings indicated that at least 21 of the 23 human recombinant CRs heteromerize with recombinant AVPR1A.

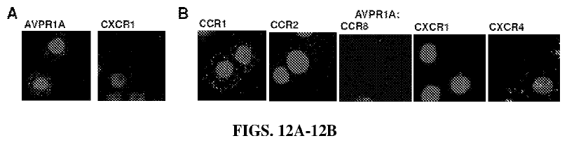

As for α1-ARs, CCR1, CCR2, CCR8 and CXCR4, AVPR1A and CXCR1 could be visualized individually in THP-1 cells in proximity ligation assays (PLA) (FIG. 12A). When PLA was performed to visualize receptor-receptor interactions, heteromers between AVPR1A and CCR1, CCR2, CCR8 and CXCR4 were detectable, whereas heteromers between AVPR1A and CXCR1 could not be detected (FIG. 12B). These findings are consistent with our observations on recombinant receptors and suggest that AVPR1A forms heteromers with most CRs that are endogenously expressed in leukocytes.

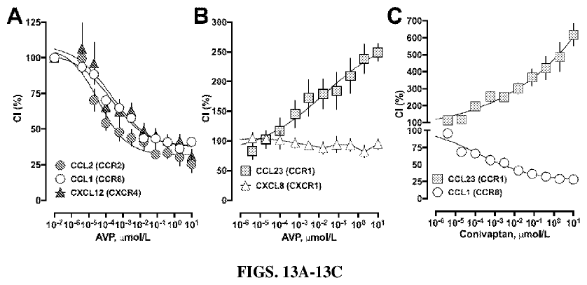

Moreover, we detected that exposure of THP-1 cells to arginine vasopressin (AVP) or the AVPR1A inhibitor Conivaptan inhibited chemotaxis mediated via the AVPR1A heteromerization partners CCR2, CCR8 and CXCR4 with high potency (IC50 37-218 pM; FIG. 13A, 13C). AVP did not affect chemotaxis mediated by CXCR1 (FIG. 13B), which does not interact with AVPR1A. Interestingly, however, AVP and Conivaptan dose-dependently enhanced chemotaxis induced via CCR1 to 250-600% of THP-1 cells not exposed to AVPR1A ligands (FIG. 13B, 13C).

### Example 3: α_{1B/D}-Adrenoceptors Regulate Chemokine Receptor-Mediated Leukocyte Migration via Formation of Heteromeric Receptor Complexes Introduction

Catecholamines regulate innate immune functions. At least 20 members of the human chemokine receptor (CR) family heteromerize with one or more members of the α₁-adrenergic receptor (AR) family in recombinant systems, and that such heteromeric complexes are detectable in human monocytes and the monocytic leukemia cell line THP-1. Ligand binding to α₁-ARs inhibited migration toward agonists of the CR heteromerization partners of α_{1B/D}-ARs with high potency and 50-77% efficacy but did not affect migration induced by a non-interacting CR. Incomplete siRNA knockdown of α1B/D-ARs in THP-1 cells partially inhibited migration toward agonists of their CR heteromerization partners. Complete α_{1B}-AR knockout via CRISPR/Cas9 gene editing in THP-1-cells (THP-1_ADRA1B^{KO}) resulted in 82% reduction of α_{1D}-AR expression and did not affect CR expression. Migration of THP-1_ADRA1B^{KO} cells toward agonists of CR heteromerization partners of α_{1B/D}-ARs was reduced by 82-95%. Findings indicate that CR:α_{1B/D}-AR heteromers contribute to normal function of the CR heteromerization partners, provide a mechanism underlying neuroendocrine control of leukocyte trafficking and offer new opportunities to modulate leukocyte and/or cancer cell trafficking in disease processes.

Most chemokine receptors (CRs) form heteromeric complexes with α₁-adrenergic receptors (ARs) in recombinant systems and that such heteromers are detectable in human monocytes and in the human monocytic leukemia cell line THP-1. Furthermore, α_{1B/D}-ARs control the function of their chemokine receptor heteromerization partners. Findings suggest that heteromeric complexes between α_{1B/D}-ARs and CRs indicate receptor heteromerization as a molecular mechanism by which stress hormones regulate leukocyte trafficking in health and disease.

Autonomic nervous systems regulate innate immune functions through the release of endogenous catecholamines, which activate the G protein-coupled receptor (GPCR) family of adrenergic receptors (ARs) expressed in the immune system (Scanzano, 2015; Pongratz, 2014; Tracel, 2009; Dhabhar, 2014; Barnes, 2015). Increased autonomic nervous system activity occurs in numerous physiological and pathological conditions, such as exercise, anxiety, trauma or infection, and has been correlated with disease activity in various autoimmune diseases (Dimitrjevic, 2012; Grisanti, 2011; Brosnan, 1985). Furthermore, evidence has been provided that catecholamines can be synthesized and released from lymphocytes, macrophages and neutrophils, which are thought to mediate para-/autocrine signaling to control leukocyte function (Bergquist, 1994; Flierl, 2007; Musso, 1996). While previous studies focused on the contribution of α₁- and α₂-Ars, the roles of α1-ARs in the regulation of immune functions are poorly understood (Grisanti, 2011). Furthermore, the detailed molecular mechanisms underlying cross-talk between the neurohormonal and innate immune system remain to be determined. The 7-transmembrane domain (7TM) protein family of chemokine receptors (CRs) is composed of 18 G protein-coupled receptors (GPCRs), 4 atypical chemokine receptors (ACKR1-4) and chemokine (C-C motif) receptor-like (CCRL)2/designated ACKR5 pending confirmation (Bachelerie, 2014; Aleander; 2017). CRs play a role in the regulation of leukocyte positioning, trafficking and recruitment, and play a role in all aspects of inflammation, including numerous disease processes, as diverse as infections, autoimmune diseases, cancer, or tissue injury and repair (Bachelerie, 2014; Olson, 2003; Busillo, 2007; Karin, 2010). All chemokine receptors except CXCR5, ACKR1 and ACKR3 have been described to be expressed by human monocytes (Hohenbaus, 2013; Tripathi, 2014; Ma, 2013; Yoshimura, 2011). α₁-ARs form hetero-oligomeric complexes with chemokine (C-X-C motif) receptor 4 (CXCR4) and atypical chemokine receptor 3 (ACKR3) in recombinant systems and in vascular smooth muscle cells, through which the receptors cross-talk (Tripathi, 2015; Albee, 2015; Gao, 2018; Evans, 2016). Similarly, recombinant CXCR2 has been reported to heteromerize with α1ₐ-AR, and the endogenously expressed receptors were found to co-localize in prostate smooth muscle (Mustafa, 2012). The existence and possible function of such heteromeric receptor complexes in human leukocytes, however, is unknown. Furthermore, it is unknown whether other members of the human CR family may also heteromerize with α1-ARs. This example evaluates the interactome between the family of human CRs and α1-ARs, and to assess the functional roles of such heteromers in the regulation of CR-mediated chemotaxis utilizing freshly isolated human monocytes and the human monocytic leukemia cell line THP-1 as model systems. The results suggest that most CRs heteromerize with α1-ARs. Moreover, α1_{B/D}-AR:CR heteromers are used for CR-mediated chemotaxis of α1_{1B/D}-AR heteromerization partners and that α1-ARs within these heteromers mediate inhibitory effects of α1-AR ligands on directed cell migration towards cognate agonists of their CR heteromerization partners.

### Results and Discussion

Bioluminescence Resonance Energy Transfer Identifies Multiple Chemokine Receptor: α1_{a/b/d}-Adrenergic Receptor Heteromers

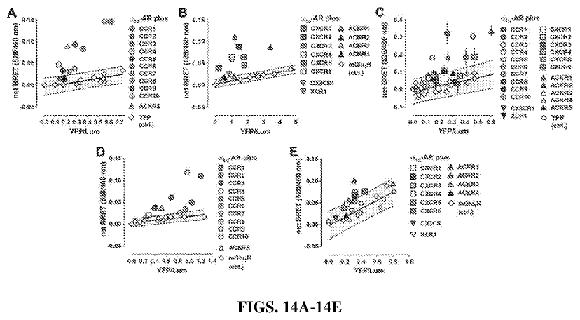

To evaluate the interactome between CRs and α1-ARs, bioluminescence resonance energy transfer (BRET) was employed to screen for interactions between α1_{a/b/d}-ARs and all 23 members of the human CR family in HEK293T cells. Cells were transfected with one α1-AR subtype C-terminally ligated to the luminescence donor Renilla Luciferase (α1_{a/b/d}-AR-Rluc) plus each of the CRs C-terminally ligated to enhanced yellow fluorescent protein (CR-YFP) in parallel and measured BRET. As a control for non-specific bystander BRET signals, cells were transfected with α1_{a/b/d}-AR-Rluc and YFP alone or with metabotropic glutamate receptor 1 (mGlu₁R)-YFP at various energy acceptor: donor ratios (Gao, 2021). FIGS. 14A-14E show the results from representative BRET screening experiments for interactions between all CRs and α1ₐ-AR (FIGS. 14A, 14B), α1_{b}-AR (FIG. 14C) and α1_{d}-AR (FIGS. 14D, 14E).

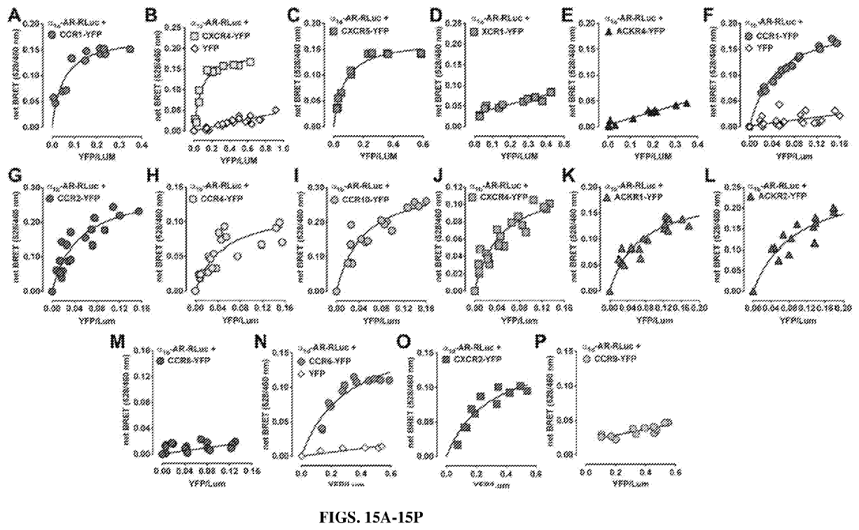

Consistent with the results from BRET screening, hyperbolic progressions of the BRET signals was observed for interactions between α1ₐ-AR and CCR1 (FIG. 15A), CXCR4 (FIG. 15B) and CXCR5 (FIG. 15C) with increasing energy acceptor:donor ratios, whereas BRET signals for interactions between α1ₐ-AR and XCR1 (FIG. 15D) or ACKR4 (FIG. 15E) were indistinguishable from non-specific bystander signals (FIG. 15B). Similarly, saturation BRET confirmed constitutive heteromerization between α1_{b}-AR and CCR1, CCR2, CCR4, CCR10, CXCR4, ACKR1 or ACKR2 (FIGS. 15F-15L) and between α1_{d}-AR and CCR6 (FIG. 15N) and CXCR2 (FIG. 15O), and non-specific interactions between α1_{b}-AR and CCRS (FIG. 15M) and between α1_{d}-AR and CCR9 (FIG. 15P). These findings validate positive and negative BRET signals for interactions between α1-ARs and CRs from our screening approach. Collectively, BRET indicates that at least 20 recombinant members of the human CR family constitutively heteromerize with at least one recombinant member of the α1-AR family.

### Chemokine Receptor: α₁-Adrenergic Receptor Heteromers are Detectable in THP-1 Cells and in Human Monocytes

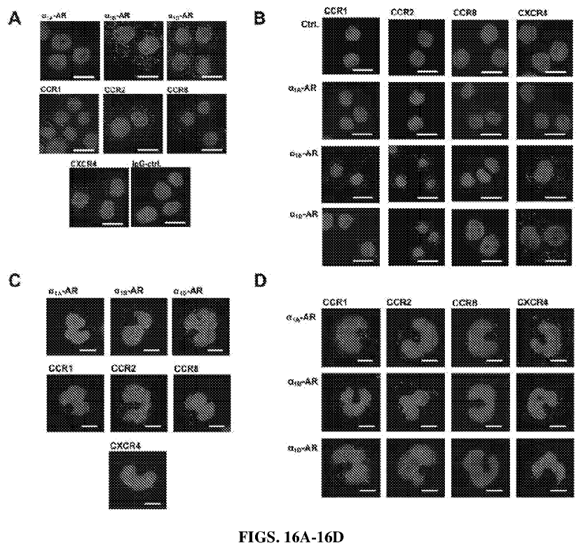

To assess whether these findings on recombinant receptors translate to endogenously expressed receptors in leukocytes, proximity ligation assays (PLA) was performed in the human monocytic leukemia cell line THP-1 and in freshly isolated human monocytes to visualize α1-ARs and selected CRs individually, and to assess α1-AR:CR interactions (Soderberg, 2006). Representative images for the detection of individual receptors and receptor-receptor interactions in THP-1 cells are shown in FIGS. 16A and 16B, respectively.

While PLA signals for α1_{A}-AR were negative in THP-1 cells, positive PLA signals for α1_{B/D}-ARs (FIG. 16A, top) were observed. Quantitative PCR confirmed that α1_{A}-AR mRNA was not detectable in THP-1 cells under cell culture conditions, whereas mRNA for all α1-AR subtypes were detectable in human primary aortic smooth muscle cells, which express all α1-AR subtypes on the cell surface (ΔCt normalized to β-actin: THP-1-α1_{A}-AR—not detectable; α1_{B}-AR—17.4; α1_{D}-AR—16.8. Human primary aortic smooth muscle cells: α1_{A}-AR—16.5; α1_{B}-AR—10.3; α1_{D}-AR—7.1). Furthermore, positive PLA signals for CCR1, CCR2, CCR8 and CXCR4 were observed in THP-1 cells. When PLA was performed to visualize receptor-receptor interactions, positive PLA signals were observed corresponding to interactions between α1_{B/D}-ARs and CCR1, CCR2 and CXCR4 (FIG. 16B). PLA signals for interactions between α1_{B/D}-ARs and CCR8 and for interactions between α1_{A}-AR and CRs were not detectable (FIG. 16B). These data agree with the expression profile of α1.AR subtypes that we determined in THP-1 cells and with the interactions between α1-ARs and CRs that we observed in BRET experiments. In contrast to THP-1 cells, all α1-AR subtypes as well as CCR1, CCR2, CCR8 and CXCR4 were detectable in freshly isolated human monocytes by PLA (FIG. 16C). Consistent with our BRET findings, positive PLA signals were observed for interactions between α1_{A}-AR and the selected CRs, and for interactions between α1_{B/D}-ARs and CCR1, CCR2 and CXCR4, but not for interactions between α1_{B/D}-ARs and CCR8 (FIG. 16D). Thus, findings suggest that positive BRET signals for recombinant receptor-receptor interactions are applicable to endogenously expressed receptors, and that such heteromeric complexes are constitutively expressed in human monocytes and THP-1 cells.

### Ligand Binding to α1_{B/D}-ARs Inhibits Chemotaxis Mediated by the Chemokine Receptor Heteromerization Partners

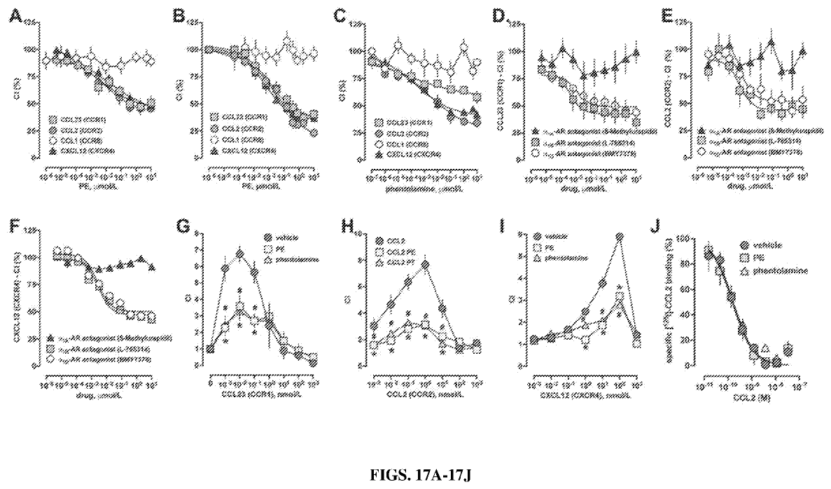

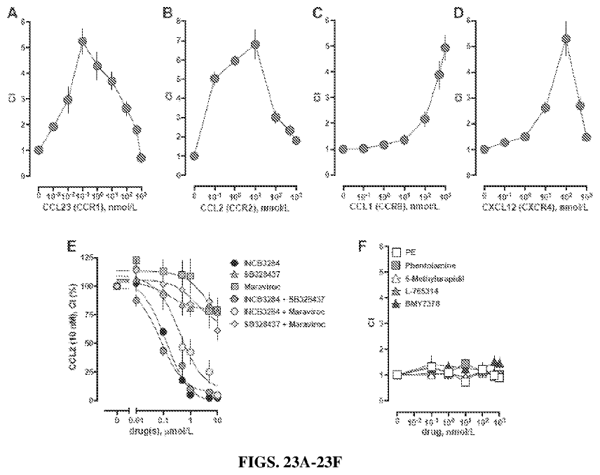

To evaluate the functional roles of α1-ARs in the regulation of CR-mediated chemotaxis, the effects of the pan-α1-AR agonist phenylephrine were tested on directed migration of THP-1 cells and human monocytes toward cognate agonists of the selected CRs in transwell migration assays. Chemotactic dose-response for each chemokine was first determined in THP-1 cells and utilized the concentrations that resulted in maximal chemotaxis for subsequent experiments (FIGS. 23A-23D). CCL23, CCL1 and CXCL12 are selective agonists for CCR1, CCR8 and CXCR4, respectively, in THP-1 cells. CCL2, however, is a cognate agonist of CCR2, CCR3 and CCR5, all of which are expressed in THP-1 cells (Martinelli, 2001; Giri, 2005). To evaluate which of these receptors mediates CCL2-induced chemotaxis, THP-1 cells were exposed to the CCR2 antagonist INCB3284, the CCR3 antagonist SB328437, the CCR5 antagonist Maraviroc or to combinations of the antagonists, and migration toward CCL2 tested. As shown in FIG. 23E, INCB3284 dose-dependently inhibited CCL2-induced chemotaxis by more than 95%. SB328437, Maraviroc or combinations of both inhibited CCL2-induced chemotaxis at the highest concentration of 10 μM by less than 30%, indicating that CCL2-induced chemotaxis in THP-1 cells is primarily mediated via CCR2. FIGS. 17A and 17B show the effects of phenylephrine on chemotaxis of THP-1 cells and human monocytes, respectively.

Cells were exposed to various concentrations of phenylephrine and their migration toward the various chemokines tested. Phenylephrine dose-dependently inhibited chemotaxis mediated via CR heteromerization partners of α1_{B/D}-ARs in THP-1 cells by 50-55% and in human monocytes by 60-77% with high potency (IC₅₀ (nM; mean±SE): THP-1 cells: CCR1—5.0±6.7; CCR2—7.5±7.7; CXCR4—17±29; human monocytes: CCR1—12±8; CCR2—37±26; CXCR4-6±3). Phenylephrine, however, did not affect chemotaxis induced by the cognate agonist of CCR8, a CR that does not heteromerize with α1_{B/D}-ARs. Because human monocytes constitutively express α1_{A}-AR and CCR8:α1_{A}-AR heteromers (FIGS. 16C, 16D), the finding that CCL1-induced chemotaxis were not affected by phenylephrine indicates distinct functional roles of α1-AR subtypes in the regulation of CR-mediated chemotaxis. While findings imply that the inhibitory effects of phenylephrine on CR-induced chemotaxis are primarily mediated via α1_{B/D}-AR, the functional role of α1_{A}-AR and the CCR8:α1_{A}-AR heteromer in human monocytes remains to be determined. Because the observed phenylephrine-mediated inhibition of chemotaxis could be a result of downstream signaling events upon activation of α1-Ars, independent of α1-AR:CR heteromerization, whether exposure of THP-1 cells to α1-AR antagonists would also affect CR-mediated chemotaxis was tested. As shown in FIG. 17C, the pan-α1-AR antagonist phentolamine dose-dependently inhibited chemotaxis mediated via CCR1, CCR2 and CXCR4 by 42%, 66% and 58%, respectively. The IC₅₀ of phentolamine for inhibition of CR-mediated chemotaxis were 0.03±0.08 nM for CCR1, and 2.7±3.5 nM and 0.2±0.2 nM for CCR2 and CXCR4, respectively. In contrast, phentolamine did not affect CCR8-mediated chemotaxis. To confirm these findings, α1-AR subtype-selective antagonists were then utilized and their effects on CCR1, CCR2 and CXCR4-mediated chemotaxis tested (FIGS. 17D-17F). In agreement with the α1-AR subtype expression in THP-1 cells, the α1_{A}-AR selective antagonist 5-Methylurapidil did not affect CR-mediated chemotaxis. In contrast, CCR1, CCR2 and CXCR4-mediated chemotaxis could be inhibited with the α1_{B}-AR selective antagonist L-765314 by 64%, 55% and 58%, respectively, and by 57%, 47% and 54%, respectively, with the α1_{D}-AR selective antagonist BMY7378. The IC₅₀ of L-765314 for inhibition of CCR1, CCR2 and CXCR4-induced chemotaxis were 0.34±0.29 nM, 0.51±0.35 nM and 2.65±0.41 nM, respectively, and 10.5±0.91 nM, 0.93±0.61 nM and 2.87±0.6 nM, respectively, for BMY7378. To exclude that the observed inhibitory effects of α1-AR ligands on migration of cells toward single optimized concentrations of chemokines are caused by a shift of their bell-shaped chemotactic dose-response curves, the chemotactic dose-responses were compared for CCL23, CCL2 and CXCL12 in the presence and absence of 10 μM of phenylephrine or phentolamine in THP-1 cells. As shown in FIG. 17G-17I, phenylephrine and phentolamine inhibited chemotaxis over the complete range of chemokine concentrations that induced chemotaxis in vehicle treated cells. Moreover, radioligand competition binding experiments (FIG. 17J) that the IC₅₀ of CCL2 to displace [¹²⁵I]-CCL2 from crude membrane preparations of THP-1 cells that were pretreated with vehicle, phenylephrine and phentolamine were indistinguishable (IC₅₀ (mean±SE, n=3):vehicle—167±23 pM; phenylephrine—150±23 pM; phentolamine—169±30 pM) and consistent with the reported affinity of CCL2 for CCR2 (Coulin, 1997; Mirzadegan, 2000; Ugoccioni, 1997). Thus, these findings suggest that the inhibitory effects of α1_{B/D}-AR ligands are primarily mediated by reducing efficacy of the CR heteromerization partners of α1_{B/D}-AR to induce chemotaxis, rather than modulating affinity for their agonists. The observations on the chemotactic behavior of normal buman monocytes and the leukemia cell line THP-1 are consistent with previous observations that phenylephrine, phentolamine, prazosin (inverse α1_{A/B/D}-AR agonist) and cyclazosin (inverse α1_{B/D}-AR agonist and α1_{A}-AR antagonist) inhibit CXCR4-mediated chemotaxis of primary human vascular smooth muscle cells (Gao, 2018; Gao et al., 2018). This indicates that the inhibitory effects of α1-AR ligands are generalizable to CR heteromerization partners of α1B/D-ARs and to other normal and malignant cell types and tissues. The findings that both agonist and antagonist binding to α1-ARs inhibited chemotaxis toward agonists of CR-heteromerization partners of α1_{B/D}-AR imply that the observed inhibitory effects are not related to downstream signaling of α1-ARs. Because none of the α1-AR ligands demonstrated chemotactic activity in THP-1 cells (FIG. 23F), their inhibitory effects on CR-mediated chemotaxis can also not be attributed to a reverse chemoattractant gradient in our experiments. However, phentolamine and all subtype selective α1-AR inhibitors that we employed in the present study are known to function as inverse agonists at α1-ARs (Kolarovszki-Sipiczki, 2007; Garcia-Sainz, 1999). This suggests that conformational changes of α1_{B/D}-AR upon ligand binding regulate the CR heteromerization partners of α1_{B/D}-AR within α1_{B/D}-AR:CR heteromeric complexes, leading to inhibition of chemokine-induced chemotaxis. Furthermore, the binding affinity of phenylephrine for α1-ARs is comparable or slightly lower than the binding affinity of endogenous catecholamines for α1-ARs (Alexander, 2017). Because the determined IC₅₀ of phenylephrine for inhibition of CR-mediated chemotaxis are in the range of physiologically and pharmacologically relevant catecholamine concentrations in humans (Dodt, 1997; Ensinger, 1992), findings suggest that circulating catecholamines in humans inhibit chemotactic activity of the cognate agonists of the CR heteromerization partners of α1_{B/D}-ARs in health and disease processes. This assumption is consistent with the significant negative correlation between endogenous norepinephrine levels and CXCL12-induced chemotaxis of peripheral blood mononuclear cells that has been observed at baseline and during acute psychological stress in healthy humans (Redwine, 2004).

### Depletion of α1_{8/D}-ARs Inhibits Chemotaxis Mediated by the Chemokine Receptor Heteromerization Partners

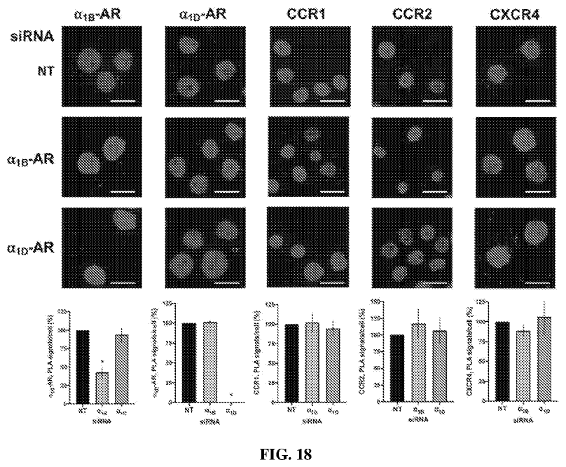

To assess the roles of heteromeric complexes between α1_{B/D}-ARs and CRs in the regulation of CR-mediated chemotaxis per se, siRNA gene silencing was utilized in THP-1 cells to deplete α1_{B/D}-AR from the cell surface. FIG. 18 shows representative images for the visualization of individual receptors after incubation of cells with non-targeting (NT) and α1_{B/D}-AR siRNA, and the quantification of PLA signals from three independent experiments.

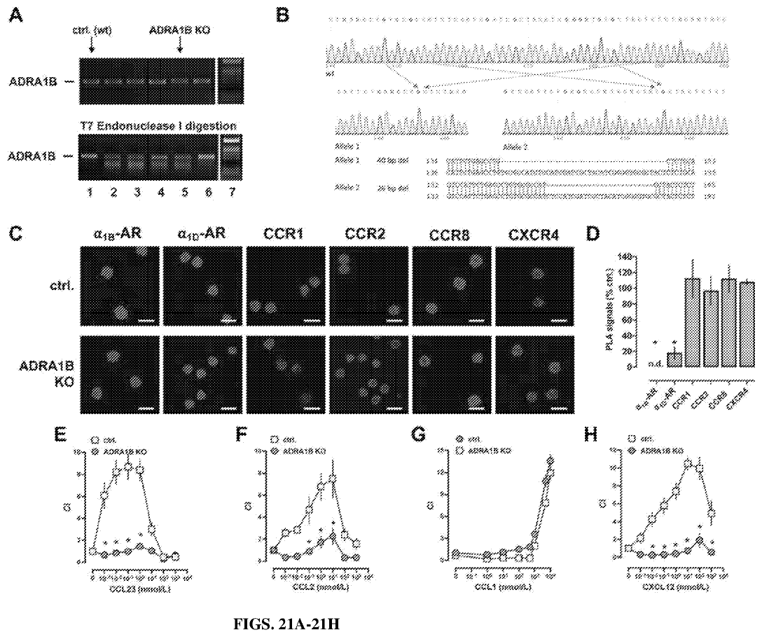

Among the clones that showed cleaved mismatch products after T7 endonuclease I digestion (lanes 2-5), lane/clone 5 was selected, DNA was PCR amplified with the ADRA1B primers and subcloned to the TA cloning vector. Sequencing of the plasmids showed a 40 bp deletion in allele 1 and a 26 bp deletion in allele 2 (FIG. 21B). Clone 5, designated THP-1_ADRA1B^{KO}, was then expanded for further experiments. FIG. 21C shows representative PLA images for the detection of individual receptors and FIG. 21D the quantification of PLA signals from three independent experiments in the THP-1 wild type clone (ctrl.) and in THP-1_ADRA1B^{KO} cells. As expected, PLA signals for α1_{B}-AR were absent in THP-1_ADRA1B^{KO} cells. When compared with control THP-1 cells, PLA signals for α1_{D}-AR were reduced by 82±7% in THP-1_ADRA1B^{KO} cells. This observation can be explained by previous findings demonstrating that quantitative translocation of α1_{D}-AR to the cell surface depends on the co-expression of α1_{B}-AR (Hague, 2004). PLA signals for CCR1, CCR2, CCR8 and CXCR4 in THP-1_ADRA1B^{KO} cells were indistinguishable from corresponding PLA signals in control THP-1 cells, which is consistent with findings after siRNA knockdown of α1_{B/D}-ARs (FIG. 18). FIGS. 21E-21H show the migration of control and THP-1_ADRA1B^{KO} cells toward agonists of the selected CRs. The chemotactic responses of THP-1_ADRA1B^{KO} cells mediated by CCR1 (FIG. 21E), CCR2 (FIG. 21F) and CXCR4 (FIG. 21H) were reduced 95%, 82% and 91%, respectively, as compared with control cells. Migration of THP-1_ADRA1B^{KO} and wild type control cells toward the CCR8 agonist was indistinguishable (FIG. 21G). These findings demonstrate that CR heteromerization partners of α1_{B/D}-ARs depend on the presence of α1_{B/D}-ARs to induce migration toward their chemokine agonists. In conclusion, the present example provides a systematic investigation of the heteromerization propensity between the GPCR families of CRs and α1-ARs. The findings suggest that at least 20 members of the human CR family can physically interact with one or more members of the α1-AR family and that such heteromers are expressed in native human monocytes and in the monocytic leukemia cell line THP-1. While the functional roles of α1_{A}-AR:CR heteromers remain to be determined, CR heteromerization partners of α1_{B/D}-AR depend on the formation of heteromeric complexes with α1_{B/D}-AR to mediate chemotactic responses toward their cognate agonists, and ligand binding to α1_{B/D}-AR within the CR:α1_{B/D}-AR heteromeric complex inhibits CR-mediated migration toward their cognate agonists with high potency. The findings provide new insights into the mechanisms by which the neuroendocrine system regulates leukocyte and/or cell migration in health and disease. The proposed mechanisms offer opportunities to modulate leukocyte or cancer cell trafficking, for example by the development of drugs that interfere with CR:α1_{B/D}-AR heteromerization or by repurposing of federal drug administration approved α1-AR ligands for the treatment of disease processes in which chemokines play a role.

### Materials and Methods

### Proteins, Antibodies and Reagents

CCL1, CCL2, N-terminally truncated CCL23 and CXCL12 were purchased from Protein Foundry (Milwaukee, WI, USA). CCL23 exists in multiple forms within the human body due to alternative splicing and post-processing; CCL23 was selected because it has been reported to bind to and activate CCR1 with higher affinity and potency than the longer CCL23 variants (Nardelli, 1999; Berkhout, 2000). [¹²⁵I]-CCL2 was purchased from Perkin Elmer (Shelton, CT, USA).

Antibodies were obtained from Abcam (Cambridge, United Kingdom: anti-α1_{A}-AR (host: rabbit, catalogue #: ab137123), anti-α1_{B}-AR (host: rabbit, catalogue #; ab169523), anti-α1_{D}-AR (host: rabbit, catalogue #: ab84402), anti-CXCR4 (host: goat, ab1670)), LifeSpan Biosciences (LSBio, Seattle, WA, USA: anti-CCR8 (host: goat, catalogue #: LS-C187704)), and R&D Systems (Minneapolis, MN, USA: anti-CCR1 (host: mouse, catalogue #: MAB145), anti-CCR2 (host: mouse, catalogue #: MAB48607), IgG isotype control (host: rabbit, catalogue #: MAB150), IgG isotype control (host: mouse, catalogue #: MAB004), IgG isotype control (host: goat, catalogue #: AB-108-C)).

Phenylephrine, phentolamine, 5′-methylurapidil, L-765314, BMY7378 and poly-1-lysine were purchased from Sigma-Aldrich (St. Louis, MO, USA). α1_{B/D}-AR siRNA, nontargeting siRNA and Accell™ transfection media were purchased from GE Dharmacon (Lafayette, CO, USA). INCB3284 (CCR2 antagonist), SB328437 (CCR3 antagonist), and Maraviroc (CCR5 antagonist) were obtained from R&D Systems. All reagents for proximity ligation assays (PLA) were from Sigma-Aldrich.

### Plasmids

cDNA for CCR1, CCR9, XCR1, ACKRI, ACKR2 and ACKR5 were obtained from Arizona State University. cDNA for all other CRs, α1_{a/b/d}-AR and mGlu₁R was from Addgene. Upper and lower case subscripts for α1-ARs are used to denote endogenous and recombinant α₁-ARs, respectively (Bylund, 1994). α1_{a/b/d}-AR subtypes and mGlu₁R were ligated at the C-termini between Age I and Xba I sites with RlucII, which was PCR amplified from CXCR4-RlucII that was kindly provided by Dr. Michel Bouvier. All CRs were ligated at the C-termini between Age I and Xba I sites with enhanced YFP. All plasmids were sequenced and verified.

### Cells and Cell Lines

The human monocytic leukemia cell line THP-1 was from American Type Culture Collection (ATCC, Manassas, VA, USA) and cultured as previously described (Saini, 2010). Briefly, cells were cultured and maintained in RPMI1640 (Sigma) supplemented with 10% fetal bovine serum, 100 U/mL penicillin and 100 ug/mL Streptomycin (Invitrogen) in 100 mm Nunc™ tissue culture dishes from ThermoFisher (Waltham, MA, USA). THP-1 cells were used at a passage number of less than ten. HEK293T cells were obtained from ATCC and maintained in Dulbecco's modified Eagle's medium (Sigma) supplemented with HyClone™ fetal bovine serum from Cytiva, 100 ∥glml penicillin (Invitrogen), and 100 μglml Streptomycin (Invitrogen) in Nunc™ 100-mm tissue culture dishes (ThermoFisher). Human monocytes were isolated from whole blood from healthy volunteers, according to our IRB protocol approved by the University of South Florida. Whole blood was drawn by venipuncture into sodium citrate CPT™ Mononuclear Cell Preparation Tubes from Becton Dickinson (Franklin Lakes, NJ, USA) and peripheral blood mononuclear cells were isolated by density gradient centrifugation at 1800 g for 20 min. CD14+/CD16− monocytes were then isolated via negative selection using MACS LS, an indirect magnetic labeling system from Miltenyi Biotech (Bergisch Gladbach, Germany). MACS LS utilizes a cocktail of biotin conjugated monoclonal anti-human antibodies against CD3, CD7, CD16, CD19, CD56, CD123, and CD235a for negative selection (catalogue #: 130-117-337). Purity, and composition of the monocyte preparations were assessed by morphology and by measuring cell size, granularity and expression of CD14/CD16 by flow cytometry.

### Gene Silencing by RNA Interference

Gene silencing with siRNA was performed as described (Tripathi, 2015; Tripathi, 2016; Saini, 2010). In brief, to deplete α1_{B/D}-ARs from the cell surface, cells were incubated in Nunc™ 6 well plates (ThermoFisher) for 3 days in Accell™ Transfection Media (GE Dharmacon) with α1_{B}-AR, α1_{D}-AR or non-targeting (negative control) siRNA (GE Dharmacon) at a concentration of 1 μM. On day 3, cells were centrifuged at 300 g for 5 min and resuspended in RPMI1640 (Sigma) supplemented with 10% HyClone™ fetal bovine serum from Cytiva (Marlborough, MA, USA), 100 μglml penicillin from Invitrogen (Waltham, MA, USA) and 100 μglml Streptomycin (Invitrogen). Cells were utilized on day 4 for experimentation.

### CRISPR/Cas9 Gene Editing

CRISPR/Cas9 gene editing was performed according to previously established criteria (Benyoucef, 2020; Baker, 2018). To generate the lentivirus encoding both sgRNA targeting α_{1B}-AR (target sequence TCCATCGATCGCTACATCG) and Cas9, 293T cells were co-transfected with the LV01 lentivirus plasmid (synthesized by Sigma) and lentiviral packing mix (Sigma) using Lipofectamine 2000 (Invitrogen). After overnight incubation, the culture medium was replaced with THP-1 medium RPMI 1640. Two days post transfection, the supernatants containing the lentiviral particles were collected and spun at 500 g for 5 min. The resultant supernatant was filtered and used to transduce THP-1 cells. Two days after transduction, cells were selected by addition of puromycin (1 μg/mL). To check the efficiency of CRISPR, the genomic DNA from transduced cells was extracted with DNAzol (Invitrogen). The DNA sequence flanking the targeting region was amplified by PCR with primers α_{1B}-F (CGCCCACCAACTACTTCATT) and α_{1B}-R (ACTCCTGCCTCTAGGTTCTT) using Platinum Blue PCR supermix (Invitrogen) according to the manufacturer's instructions. The PCR product was examined for mutations with the T7 endonuclease I (T7EI) assay kit (IDT) following the manufacturer's instructions. After confirming efficient editing of α_{1B}-AR genomic DNA, the transduced cells were replated in 96-well plates at 1 cell/well in the conditioned medium containing 20% FBS. Three weeks later, clones were replated in 24-well plates and subjected to screening with the T7 endonuclease I (T7EI) assay kit. To detect the sequences of both alleles, DNAs from the edited clones were PCR amplified with the above primers and subcloned to the TA cloning vector. The resulting plasmids were sequenced. The clones containing out of frame inserts or deletions in both alleles (designated THP-1_ADRA1B^{KO}) were expanded for experiments.

### Bioluminescence Resonance Energy Transfer (BRET)

BRET assays were performed in human embryonic kidney (HEK293T) cells as described previously (Gao, 2021; Gao, 2020, Characterization, 2020; Gao, Regulation, 2020; Albee, 2018). HEK293T cells were seeded in 12-well plates and transfected with the indicated plasmids using Lipofectamine 3000 as a transfection reagent (Thermo Fisher). For BRET screening assays, α1_{a/b/d}-RLuc was transfected at a fixed amount of 5 ng alone, with increasing amounts of YFP or mGlu1R YFP or a fixed amount of 25 ng CR-YFP; for saturation BRET experiments α1_{a/b/d}-RLuc was transfected at a fixed amount of 5 ng with increasing amounts of CR-YFP. In all assays, empty vector pcDNA3.1 was added to keep the total amount DNA for each transfection constant. Cells were incubated overnight and subsequently replated to poly-l-lysine (Sigma) coated 96-well white plates (Greiner Bio-One, Frickenhausen, Germany) and incubated again overnight. Cells were then washed with PBS, and fluorescence was measured in a plate reader (Cytation 1 Cell Imaging Multi-Mode Reader, BioTek, Winooski, VT, US; λ_{excitation} 485 nm, λₑₘᵢₛₛᵢₒₙ 528 nm). For BRET measurements, coelenterazine H was added at a final concentration of 5 μM. After 10 min incubation at room temperature, luminescence was measured at 460 nm and 528 nm. The BRET signal was calculated as the ratio of the relative luminescence units (RLU) measured at 528 nm over RLU at 460 nm. The net BRET was calculated by subtracting the BRET signal detected when α1_{a/b/d}-RLuc was transfected alone. For screening and saturation experiments, net BRET ratios are expressed as a function of fluorescence/total luminescence.

### Proximity Ligation Assay (PLA)

PLAs were performed as previously described (Tripathi, 2015; Albee, 2017; Evans, 2016; Albee, 2018). In brief, THP-1 cells were deposited in a monolayer on glass slides (ThermoFisher) by centrifugation at 800 g using a Cytospin 4 Centrifuge (ThermoFisher). Cell monolayers were isolated into individual wells using a water repellent solution (super PAP pen, ThermoFisher). Subsequently, cells were fixed with 4% (wt/vol) paraformaldehyde for 15 min at room temperature and then blocked overnight at 4° C. with Sigma-Aldrich Duolink® PLA blocking reagent. Blocked slides were incubated with indicated primary antibody(s) in dilutions of 1 μg/mL corresponding to the receptor(s) of interest. IgG isotype antibodies were utilized as a control. Slides were subsequently washed with PBS and incubated (60 min at 37° C. in a humidifying chamber) with secondary species-specific antibodies conjugated to plus and minus PLA probes (1:5). Probed slides were then washed with Sigma-Aldrich Duolink® wash buffer A and incubated with ligation reagent (30 min at 37° C. in a humidifying chamber). After ligation, slides were washed with wash buffer A again and then incubated with amplification reagent (105 min at 37° C. in a bumidifying chamber). Slides were then washed twice with wash buffer B and then once with 0.01× wash buffer B in ddH₂O and allowed to dry. Treated slides were mounted with 50 μL per well of Duolink® in situ mounting medium with [symbol] , -diamidino-2-phenylindole (DAPI) overnight at −20° C. PLA signals (Duolink® in situ detection reagents red [λ_{excitation/emission} 598/634 nm]) were identified as red fluorescent spots under a Keyence (Osaka, Japan) BZ-X710 fluorescence microscope [60×/1.50 oil] at room temperature. PLA signals were quantified using Image J (National Institutes of Health). Images were imported in merged.tiff formats containing both signal and nuclei channels. Merged images were visually verified for analytical quality. Comparisons and statistical analyses were performed only when PLA assays were performed on the same day in parallel experiments. Fluorescence microscopy was performed with the identical settings. For each experiment and condition, 10 randomly selected non-overlapping vision fields were analyzed.

### Chemotaxis Assays

Cell migration was measured employing the Neuroprobe (Gaithersburg, MD, USA) ChemoTx® Disposable Chemotaxis System. A 96-well Boyden-chamber (30 μL/well, 8 μm pores, 1×10⁵ pores/cm² pore density) was selected for all chemotaxis experiments. Bottom wells of the Boyden-chamber were loaded with 30 μL of test substances at various concentrations. Top wells of the membrane were loaded with 25 μL of THP-1 cells, THP-1_ADRA1B^{KO}, wild type THP-1 cells after clonal expansion (=control for THP-1_ADRA1B^{KO}) or freshly isolated monocytes (200 cells/μL). THP-1 cells were suspended in depleted RPMI1640 (0.5% HyClone™ fetal bovine serum, Sigma); Freshly isolated monocytes were suspended in RPMI1640 (0.5% human platelet poor plasma, Sigma). After 3 hours, transmigrated cells were counted utilizing the Cytation 1 plate reader (BioTek) by direct imaging in high contrast bright field (4×) and post-imaging particle analyses with Gen5 (v3.05) Imaging & Microscopy Software (BioTek). The chemotactic index (CI) was calculated as the ratio of cells that transmigrated in the presence versus the absence of the test solutions.

### Quantitative PCR

Total RNA was extracted from cells using TRizol from Invitrogen. RNA was reverse-transcribed to cDNAs with Applied BiosystemsM High-Capacity cDNA Reverse Transcription Kit (Themofisher) following the manufacturer manual. Premixed primers and probes for α_{1A}-AR, α_{1B}-AR, α_{1D}-AR and β-actin were synthesized by IDT. Quantitative PCR was performed with the Bio-Rad CFX Connect Real Time PCR System.

### Radioligand Competition Binding Assays

THP-1 cells were incubated with vehicle, 10 μM of phenylephrine or 10 μM of phentolamine (10⁸ cells, 30 min at 37° C.) in cell culture medium, cooled on ice for 5 min, centrifuged (300× g, 4° C.), wasbed with 20 mL cold phosphate buffered saline and resuspended in 10 mL of hypotonic buffer (10 mM HEPES, 0.2 mM CaCl₂, 1 mM MgCl₂, 0.02% BSA, pH 7.2). Cells were then centrifuged at 30,000× g (20 min, 4° C.), the supernatant was discarded, and pelleted cell fragments were snap-frozen in liquid nitrogen. The cell pellets were resuspended in 50 mM HEPES, 5 mM MgCl₂, 1 mM CaCl₂, 0.1% w/v BSA, pH 7.2, and then incubated with 50 pM [¹²⁵I]-CCL2, 1× Xpert Protease Inhibitor Cocktail (GenDEPOT), and varying concentrations of CCL2 for 1.5 h at 25° C. Bound [¹²⁵I]-CCL2 was collected via vacuum filtration using buffer containing 10 mM HEPES (pH 7.4) and 0.5 M NaCl at 4° C., and a cell harvester (Brandel) equipped with glass fiber filters. Radioactivity was measured in a gamma counter at 70% efficiency.

### Data Analyses

Data are expressed as mean±standard error from n independent experiments that were performed on different days or as mean±standard deviation from triplicate measurements for representative BRET screening experiments. Data were analyzed using GraphPad Prism v. 9.02 software. BRET and dose-response curves were analyzed using nonlinear regression analyses. Unpaired Student's t-test, one-way analyses of variance (ANOVA) with Dunnett's multiple comparisons test and two-way ANOVA with Dunnett's multiple comparisons test were used as appropriate. A two-tailed p<0.05 was considered significant.

Other advantages which are obvious, and which are inherent to the invention, will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

### REFERENCES

A. Scanzano, M. Cosentino, Adrenergic regulation of innate immunity: a review. Front Pharmacol 6, 171 (2015).
G. Pongratz, R. H. Straub, The sympathetic nervous response in inflammation. Arthritis Res Ther 16, 504 (2014).
K. J. Tracey, Reflex control of immunity. Nat Rev Immunol 9, 418-428 (2009).
F. S. Dhabhar, Effects of stress on immune function: the good, the bad, and the beautiful. Immunol Res 58, 193-210 (2014).
M. A. Barnes, M. J. Carson, M. G. Nair, Non-traditional cytokines: How catecholamines and adipokines influence macrophages in immunity, metabolism and the central nervous system. Cytokine 72, 210-219 (2015).
M. Dimitrijevic, S. Stanojevic, N. Kustrimovic, G. Leposavic, End-point effector stress mediators in neuroimmune interactions: their role in immune system homeostasis and autoimmune pathology. Immunol Res 52, 64-80 (2012).
L. A. Grisanti et al., alphα1-adrenergic receptors positively regulate Toll-like receptor cytokine production from human monocytes and macrophages. J Pharmacol Exp Ther 338, 648-657 (2011).
C. F. Brosnan et al., Prazosin, an alpha 1-adrenergic receptor antagonist, suppresses experimental autoimmune encephalomyelitis in the Lewis rat. Proc Natl Acad Sci USA 82, 5915-5919 (1985).
J. Bergquist, A. Tarkowski, R. Ekman, A. Ewing, Discovery of endogenous catecholamines in lymphocytes and evidence for catecholamine regulation of lymphocyte function via an autocrine loop. Proc Natl Acad Sci USA 91, 12912-12916 (1994).
M. A. Flierl et al., Phagocyte-derived catecholamines enhance acute inflammatory injury. Nature 449, 721-725 (2007).
M. A. Flierl et al., Upregulation of phagocyte-derived catecholamines augments the acute inflammatory response. PLoS One 4, e4414 (2009).
N. R. Musso, S. Brenci, M. Setti, F. Indiveri, G. Lotti, Catecholamine content and in vitro catecholamine synthesis in peripheral human lymphocytes. J Clin Endocrinol Metab 81, 3553-3557 (1996).
L. A. Grisanti, D. M. Perez, J. E. Porter, Modulation of immune cell function by alpha(1)-adrenergic receptor activation. Curr Top Membr 67, 113-138 (2011).
F. Bachelerie et al., International Union of Pharmacology. LXXXIX. Update on the extended family of chemokine receptors and introducing a new nomenclature for atypical chemokine receptors. Pharmacological reviews 66, 1-79 (2014).
S. P. Alexander et al., THE CONCISE GUIDE TO PHARMACOLOGY 2017/18: G protein-coupled receptors. Br J Pharmacol 174 Suppl 1, S17-S129 (2017).
T. S. Olson, K. Ley, Chemokines and chemokine receptors in leukocyte trafficking. Am J Physiol Regul Integr Comp Physiol 283, R7-28 (2002).
J. M. Busillo, J. L. Benovic, Regulation of CXCR4 signaling. Biochim Biophys Acta 1768, 698, 952-963 (2007).
N. Karin, The multiple faces of CXCL12 (SDF-1alpha) in the regulation of immunity during health and disease. Journal of leukocyte biology 88, 463-473 (2010).
D. M. Hohenhaus et al., An mRNA atlas of G protein-coupled receptor expression during primary human monocyte/macrophage differentiation and lipopolysaccharide-mediated activation identifies targetable candidate regulators of inflammation. Immunobiology 218, 1345-1353 (2013).
A. Tripathi, J. D. Davis, D. M. Staren, B. F. Volkman, M. Majetschak, CXC chemokine receptor 4 signaling upon co-activation with stromal cell-derived factor-1alpha and ubiquitin. Cytokine 65, 121-125 (2014).
W. Ma, Y. Liu, N. Ellison, J. Shen, Induction of C-X-C Chemokine Receptor Type 7 (CXCR7) Switches Stromal Cell-Derived Factor-1 (SDF-1) Signaling and Phagocytic Activity in Macrophages Linked to Atherosclerosis. The Journal of biological chemistry 10.1074/jbc.M112.445510 (2013).
T. Yoshimura, J. J. Oppenheim, Chemokine-like receptor 1 (CMKLR1) and chemokine (C-C motif) receptor-like 2 (CCRL2); two multifunctional receptors with unusual properties. Exp Cell Res 317, 674-684 (2011).
A. Tripathi et al., Heteromerization of chemokine (C-X-C motif) receptor 4 with alphα1 A/B-adrenergic receptors controls alphα1-adrenergic receptor function. Proc Natl Acad Sci USA 112, E1659-1668 (2015).
L. J. Albee et al., alphα1-Adrenergic Receptors Function Within Hetero-Oligomeric Complexes With Atypical Chemokine Receptor 3 and Chemokine (C-X-C motif) Receptor 4 in Vascular Smooth Muscle Cells. J Am Heart Assoc 6, e006575 (2017).
X. Gao, L. J. Albec, B. F. Volkman, V. Gaponenko, M. Majetschak, Asymmetrical ligand-induced cross-regulation of chemokine (C-X-C motif) receptor 4 by alphα1-adrenergic receptors at the heteromeric receptor complex. Sci Rep 8, 2730 (2018).
A. E. Evans et al., New Insights into Mechanisms and Functions of Chemokine (C-X-C Motif) Receptor 4 Heteromerization in Vascular Smooth Muscle. Int J Mol Sci 17, 971 (2016). 27.
S. Mustafa et al., Identification and profiling of novel alphα1A-adrenoceptor-CXC chemokine receptor 2 heteromer. The Journal of biological chemistry 287, 12952-12965(2012).
X. Gao, G. A. Enten, A. J. DeSantis, M. Majetschak, Class A G protein-coupled receptors assemble into functional higher-order hetero-oligomers. FEBS Lett 595, 1863-1875 (2021).
O. Soderberg et al., Direct observation of individual endogenous protein complexes in situ by proximity ligation. Nature methods 3, 995-1000 (2006).
A. Tripathi, V. Gaponenko, M. Majetschak, Commercially available antibodies directed against alpha-adrenergic receptor subtypes and other G protein-coupled receptors with acceptable selectivity in flow cytometry experiments. Naunyn Schmiedebergs Arch Pharmacol 389, 243-248 (2016).
R. Martinelli, I. Sabroe, G. LaRosa, T. J. Williams, J. E. Pease, The CC chemokine eotaxin (CCL11) is a partial agonist of CC chemokine receptor 2b. J Biol Chem 276, 42957-42964 (2001).
R. K. Giri, V. Rajagopal, S. Shahi, B. V. Zlokovic, V. K. Kalra, Mechanism of amyloid peptide induced CCR5 expression in monocytes and its inhibition by siRNA for Egr-1. Am J Physiol Cell Physiol 289, C264-276 (2005).
F. Coulin et al., Characterisation of macrophage inflammatory protein-5/human CC cytokine-2, a member of the macrophage-inflammatory-protein family of chemokines. Eur J Biochem 248, 507-515 (1997).
T. Mirzadegan et al., Identification of the binding site for a novel class of CCR2b chemokine receptor antagonists; binding to a common chemokine receptor motif within the helical bundle. J Biol Chem 275, 25562-25571 (2000).
M. Uguccioni et al., High expression of the chemokine receptor CCR3 in human blood basophils. Role in activation by eotaxin, MCP-4, and other chemokines. J Clin Invest 100, 1137-1143 (1997).
X. Gao et al., Partial agonist activity of alphα1-adrenergic receptor antagonists for chemokine (C-X-C motif) receptor 4 and atypical chemokine receptor 3. PLoS One 13, e0204041 (2018).
Z. Kolarovszki-Sipiczki et al., Effect of alpha-adrenoceptor subtype-selective inverse agonists on non-pregnant and late-pregnant cervical resistance in vitro in the rat. Clin Exp Pharmacol Physiol 34, 42-47 (2007).
J. A. Garcia-Sainz, M. E. Torres-Padilla, Modulation of basal intracellular calcium by inverse agonists and phorbol myristate acetate in rat-1 fibroblasts stably expressing alphα1d-adrenoceptors. FEBS Lett 443, 277-281 (1999).
C. Dodt, U. Breckling, I. Derad, H. L. Fehm, J. Born, Plasma epinephrine and norepinephrine concentrations of healthy humans associated with nighttime sleep and morning arousal. Hypertension 30, 71-76 (1997).
H. Ensinger, B. Stein, O. Jager, A. Grunert, F. W. Ahnefeld, Relationship between infusion rates, plasma concentrations, and cardiovascular and metabolic effects during the infusion of norepinephrine in healthy volunteers. Crit Care Med 20, 1250-1256 (1992).
L. Redwine et al., Differential immune cell chemotaxis responses to acute psychological stress in Alzheimer caregivers compared to non-caregiver controls. Psychosom Med 66, 770-775 (2004).
M. A. Uberti, R. A. Hall, K. P. Minneman, Subtype-specific dimerization of alpha 1-adrenoceptors: effects on receptor expression and pharmacological properties. Mol Pharmacol 64, 1379-1390 (2003).
L. J. Albee, X. Gao, M. Majetschak, Plasticity of seven-transmembrane-helix receptor heteromers in human vascular smooth muscle cells. PLoS One 16, e0253821 (2021).
C. Hague et al., Heterodimers of alphα1B- and alphα1D-adrenergic receptors form a single functional entity. Mol Pharmacol 69, 45-55 (2006).
C. Hague, M. A. Uberti, Z. Chen, R. A. Hall, K. P. Minneman, Cell surface expression of alphα1D-adrenergic receptors is controlled by heterodimerization with alphα1B-adrenergic receptors. J Biol Chem 279, 15541-15549 (2004).
B. Nardelli et al., Characterization of the signal transduction pathway activated in human monocytes and dendritic cells by MPIF-1, a specific ligand for CC chemokine receptor 1. J Immunol 162, 435-444 (1999).
T. A. Berkhout et al., Selective binding of the truncated form of the chemokine CKbeta8 (25-99) to CC chemokine receptor 1 (CCR1). Biochem Pharmacol 59, 591-596 (2000).
D. B. Bylund et al., International Union of Pharmacology nomenclature of adrenoceptors. Pharmacol Rey 46, 121-136 (1994).
V. Saini, A. Marchese, M. Majetschak, CXC chemokine receptor 4 is a cell surface receptor for extracellular ubiquitin. J Biol Chem 285, 15566-15576 (2010).
A. Benyoucef, L. Marchitto, F. Touzot, CRISPR gene-engineered CYBB(ko) THP-1 cell lines highlight the crucial role of NADPH-induced reactive oxygen species for regulating inflammasome activation. J Allergy Clin Immunol 145, 1690-1693 e1695 (2020).
P. J. Baker, S. L. Masters, Generation of Genetic Knockouts in Myeloid Cell Lines Using a Lentiviral CRISPR/Cas9 System. Methods Mol Biol 1714, 41-55 (2018).
X. Gao et al., Characterization of heteromeric complexes between chemokine (C-X-C motif) receptor 4 and alphα1-adrenergic receptors utilizing intermolecular bioluminescence resonance energy transfer assays. Biochem Biophys Res Commun 528, 368-375 (2020).
X. Gao et al., Regulation of the thrombin/protease-activated receptor 1 axis by chemokine (CXC motif) receptor 4. J Biol Chem 295, 14893-14905 (2020).
L. J. Albee et al., Identification and functional characterization of arginine vasopressin receptor 1A: atypical chemokine receptor 3 heteromers in vascular smooth muscle. Open Biol 8, 170207 (2018).

### Gomes, I., Ayoub, M. A., Fujita, W., Jaeger, W. C., Pfleger, K. D. & Devi, L. A. (2016) G Protein-Coupled Receptor Heteromers, Annu Rev Pharmacol Toxicol. 56, 403-25.

Farran, B. (2017) An update on the physiological and therapeutic relevance of GPCR oligomers, Pharmacol Res. 117, 303-327.
Dale, N. C., Johnstone, E. K. M. & Pfleger, K. D. G. (2022) GPCR heteromers: An overview of their classification, function and physiological relevance, Frontiers in Endocrinology. 13.
Ferre, S., Baler, R., Bouvier, M., Caron, M. G., Devi, L. A., Durroux, T., Fuxe, K., George, S. R., Javitch, J. A., Lohse, M. J., Mackie, K., Milligan, G., Pfleger, K. D., Pin, J. P., Volkow, N. D., Waldhoer, M., Woods, A. S. & Franco, R. (2009) Building a new conceptual framework for receptor heteromers, Nat Chem Biol. 5, 131-4.

Ferre, S., Casado, V., Devi, L. A., Filizola, M., Jockers, R., Lohse, M. J., Milligan, G., Pin, J. P. & Guitart, X. (2014) G protein-coupled receptor oligomerization revisited: functional and pharmacological perspectives, Pharmacol Rev. 66, 413-34.

Ferre, S., Ciruela, F., Casado, V. & Pardo, L. (2020) Oligomerization of G protein-coupled receptors: Still doubted?, Prog Mol Biol Transl Sci. 169, 297-321.

### Franco, R., Martinez-Pinilla, E., Lanciego, J. L. & Navarro, G. (2016) Basic Pharmacological and Structural Evidence for Class A G-Protein-Coupled Receptor Heteromerization, Front Pharmacol. 7, 76.

Lambert, N. A. (2010) GPCR dimers fall apart, Sci Signal. 3, pe12.
Tripathi, A., Vana, P. G., Chavan, T. S., Brueggemann, L. I., Byron, K. L., Tarasova, N. I., Volkman, B. F., Gaponenko, V. & Majetschak, M. (2015) Heteromerization of chemokine (C-X-C motif) receptor 4 with alpha1A/B-adrenergic receptors controls alpha1-adrenergic receptor function, Proc Natl Acad Sci USA. 112, E1659-68.
Evans, A. E., Tripathi, A., LaPorte, H. M., Brueggemann, L. I., Singh, A. K., Albee, L. J., Byron, K. L., Tarasova, N. I., Volkman, B. F., Cho, T. Y., Gaponenko, V. & Majetschak, M. (2016) New Insights into Mechanisms and Functions of Chemokine (C-X-C Motif) Receptor 4 Heteromerization in Vascular Smooth Muscle, Int J Mol Sci. 17, 971.

Albee, L. J., Eby, J. M., Tripathi, A., LaPorte, H. M., Gao, X., Volkman, B. F., Gaponenko, V. & Majetschak, M. (2017) alpha1-Adrenergic Receptors Function Within Hetero-Oligomeric Complexes With Atypical Chemokine Receptor 3 and Chemokine (C-X-C motif) Receptor 4 in Vascular Smooth Muscle Cells, J Am Heart Assoc. 6, e006575.

Albee, L. J., Gao, X. & Majetschak, M. (2021) Plasticity of seven-transmembrane-helix receptor heteromers in human vascular smooth muscle cells, PLoS One. 16, e0253821.
Gao, X., Albee, L. J., Volkman, B. F., Gaponenko, V. & Majetschak, M. (2018) Asymmetrical ligand-induced cross-regulation of chemokine (C-X-C motif) receptor 4 by alpha1-adrenergic receptors at the heteromeric receptor complex, Sci Rep. 8, 2730.
Gao, X., DeSantis, A. J., Enten, G. A., Weche, M., Marcet, J. E. & Majetschak, M. (2022) Heteromerization between alpha1B-adrenoceptor and chemokine (C-C motif) receptor 2 biases alpha1B-adrenoceptor signaling: Implications for vascular function, FEBS Lett, Online ahead of print.
Gao, X., Enten, G. A., DeSantis, A. J. & Majetschak, M. (2021) Class A G protein-coupled receptors assemble into functional higher-order hetero-oligomers, FEBS Lett. 595, 1863-1875.
Gao, X., Enten, G. A., DeSantis, A. J., Volkman, B. F., Gaponenko, V. & Majetschak, M. (2020) Characterization of heteromeric complexes between chemokine (C-X-C motif) receptor 4 and alpha1-adrenergic receptors utilizing intermolecular bioluminescence resonance energy transfer assays, Biochem Biophys Res Commun. 528, 368-375.
Enten, G. A., Gao, X., Strzelinski, H. R., Weche, M., Liggett, S. B. & Majetschak, M. (2022) alpha1B/D-adrenoceptors regulate chemokine receptor-mediated leukocyte migration via formation of heteromeric receptor complexes, Proc Natl Acad Sci USA. 119, e2123511119.
Bylund, D. B., Eikenberg, D. C., Hieble, J. P., Langer, S. Z., Lefkowitz, R. J., Minneman, K. P., Molinoff, P. B., Ruffolo, R. R., Jr. & Trendelenburg, U. (1994) International Union of Pharmacology nomenclature of adrenoceptors, Pharmacol Rev. 46, 121-36.
Olsen, R. H. J., DiBerto, J. F., English, J. G., Glaudin, A. M., Krumm, B. E., Slocum, S. T., Che, T., Gavin, A. C., McCorvy, J. D., Roth, B. L. & Strachan, R. T. (2020) TRUPATH, an open-source biosensor platform for interrogating the GPCR transducerome, Nat Chem Biol. 16, 841-849.
Albee, L. J., LaPorte, H. M., Gao, X., Eby, J. M., Cheng, Y. H., Nevins, A. M., Volkman, B. F., Gaponenko, V. & Majetschak, M. (2018) Identification and functional characterization of arginine vasopressin receptor 1A: atypical chemokine receptor 3 heteromers in vascular smooth muscle, Open Biol. 8, 170207.
Gao, X., Cheng, Y. H., Enten, G. A., DeSantis, A. J., Gaponenko, V. & Majetschak, M. (2020) Regulation of the thrombin/protease-activated receptor 1 axis by chemokine (CXC motif) receptor 4, J Biol Chem. 295, 14893-14905.
Eby, J. M., Abdelkarim, H., Albee, L. J., Tripathi, A., Gao, X., Volkman, B. F., Gaponenko, V. & Majetschak, M. (2017) Functional and structural consequences of chemokine (C-X-C motif) receptor 4 activation with cognate and non-cognate agonists, Mol Cell Biochem. 434, 143-151.
Cheng, Y. H., Eby, J. M., LaPorte, H. M., Volkman, B. F. & Majetschak, M. (2017) Effects of cognate, non-cognate and synthetic CXCR4 and ACKR3 ligands on human lung endothelial cell barrier function, PLoS One. 12, e0187949.
Armando, S., Quoyer, J., Lukashova, V., Maiga, A., Percherancier, Y., Heveker, N., Pin, J. P., Prezeau, L. & Bouvier, M. (2014) The chemokine CXC4 and CC2 receptors form homo- and heterooligomers that can engage their signaling G-protein effectors and betaarrestin, FASEB J. 28, 4509-23.
Berchiche, Y. A., Gravel, S., Pelletier, M. E., St-Onge, G. & Heveker, N. (2011) Different effects of the different natural CC chemokine receptor 2b ligands on beta-arrestin recruitment, Galphai signaling, and receptor internalization, Mol Pharmacol. 79, 488-98.
Kroeze, W. K., Sassano, M. F., Huang, X. P., Lansu, K., McCorvy, J. D., Giguere, P. M., Sciaky, N. & Roth, B. L. (2015) PRESTO-Tango as an open-source resource for interrogation of the druggable human GPCRome, Nat Struct Mol Biol. 22, 362-9.
Martinelli, R., Sabroe, I., LaRosa, G., Williams, T. J. & Pease, J. E. (2001) The CC chemokine eotaxin (CCL11) is a partial agonist of CC chemokine receptor 2b, J Biol Chem. 276, 42957-64.
Giri, R. K., Rajagopal, V., Shahi, S., Zlokovic, B. V. & Kalra, V. K. (2005) Mechanism of amyloid peptide induced CCR5 expression in monocytes and its inhibition by siRNA for Egr-1, Am J Physiol Cell Physiol. 289, C264-76.
Napier, C., Sale, H., Mosley, M., Rickett, G., Dorr, P., Mansfield, R. & Holbrook, M. (2005) Molecular cloning and radioligand binding characterization of the chemokine receptor CCR5 from rhesus macaque and human, Biochem Pharmacol. 71, 163-72.
Daugherty, B. L., Siciliano, S. J., DeMartino, J. A., Malkowitz, L., Sirotina, A. & Springer, M. S. (1996) Cloning, expression, and characterization of the human eosinophil eotaxin receptor, J Exp Med. 183, 2349-54.
Coulin, F., Power, C. A., Alouani, S., Peitsch, M. C., Schroeder, J. M., Moshizuki, M., Clark-Lewis, I. & Wells, T. N. (1997) Characterisation of macrophage inflammatory protein-5/human CC cytokine-2, a member of the macrophage-inflammatory-protein family of chemokines, Eur J Biochem. 248, 507-15.
Luttrell, L. M., Wang, J., Plouffe, B., Smith, J. S., Yamani, L., Kaur, S., Jean-Charles, P. Y., Gauthier, C., Lee, M. H., Pani, B., Kim, J., Ahn, S., Rajagopal, S., Reiter, E., Bouvier, M., Shenoy, S. K., Laporte, S. A., Rockman, H. A. & Lefkowitz, R. J. (2018) Manifold roles of beta-arrestins in GPCR signaling elucidated with siRNA and CRISPR/Cas9, Sci Signal. 11.
Xue, C. B., Feng, H., Cao, G., Huang, T., Glenn, J., Anand, R., Meloni, D., Zhang, K., Kong, L., Wang, A., Zhang, Y., Zheng, C., Xia, M., Chen, L., Tanaka, H., Han, Q., Robinson, D. J., Modi, D., Storace, L., Shao, L., Sharief, V., Li, M., Galya, L. G., Covington, M., Scherle, P., Diamond, S., Emm, T., Yeleswaram, S., Contel, N., Vaddi, K., Newton, R., Hollis, G., Friedman, S. & Metcalf, B. (2011) Discovery of INCB3284, a Potent, Selective, and Orally Bioavailable hCCR2 Antagonist, ACS Med Chem Lett. 2, 450-4.
Xiu, F., Stanojcic, M. & Jeschke, M. G. (2013) Norepinephrine inhibits macrophage migration by decreasing CCR2 expression, PLoS One. 8, e69167.
Shenoy, S. K. & Lefkowitz, R. J. (2011) beta-Arrestin-mediated receptor trafficking and signal transduction, Trends Pharmacol Sci. 32, 521-33.
Mahalingam, S. & Karupiah, G. (1999) Chemokines and chemokine receptors in infectious diseases, Immunol Cell Biol. 77, 469-75.
Costa, M. J., Kudaravalli, J., Liu, W. H., Stock, J., Kong, S. & Liu, S. H. (2018) A mouse model for evaluation of efficacy and concomitant toxicity of anti-human CXCR4 therapeutics, PLoS One. 13, e0194688.
UniProt, C. (2021) UniProt: the universal protein knowledgebase in 2021, Nucleic Acids Res. 49, D480-D489.

1. A method of modulating inflammation, comprising: administering to a subject in need thereof a therapeutically effective amount of a modulator of an adrenergic receptor and/or an arginine vasopressin receptor.

2. A method of modulating cancer cell trafficking, comprising: administering to a subject in need thereof a therapeutically effective amount of a modulator of an adrenergic receptor and/or an arginine vasopressin receptor.

3. (canceled)

4. A method of modulating activity of a chemokine receptor, comprising:
administering to a subject in need thereof a therapeutically effective amount of a modulator of an adrenergic receptor and/or an arginine vasopressin receptor.

5. The method of claim 4, wherein the chemokine receptor is CCR1, CCR2, CCR3, CCR4, CCR5, CCR6, CCR7, CCR8, CCR9, CCR10, CXCR1, CXCR2, CXCR3, CXCR4, CXCR5, CXCR6, ACKR1, ACKR2, ACKR3, ACKR4, ACKR5, or CX3CR.

6. The method of claim 1, wherein the adrenergic receptor is alpha1-adregeneric receptor or wherein the arginine vasopressin receptor is arginine vasopressin receptor 1A.

7. The method of claim 1, wherein the modulator of an adrenergic receptor is an antagonist selected from the group consisting of esmolol, betaxolol, metoprolol, dapiprazole, atenolol, alfuzosin, mirtazapine, timolol, profenamine, prazosin, sotalol, carteolol, propranolol, doxazosin, labetalol, bisoprolol, phentolamine, nicergoline, tamsulosin, tolazoline, alprenolol, quinidine, phenoxybenzamine, pindolol, ergoloid mesylate, carvedilol, bretylium, terazosin, acebutolol, nadolol, levobunolol, metipranolol, bevantolo, practolol, penbutolol, yohimbine, oxprenolol, 1-benzylimidazole, celiprolol, silodosin, esmirtazapine, bufuralol, bopindolol, bupranolol, lurasidone, indoramin, indenolol, ifenprodil, befunolol, arotinolol, moxisylyte, trimazosin, atipamezole, talinolol, naftopidil, landiolol, bunazosin, idazoxan, urapidil, bucindolol, dihydroergocristine, cloranolol, mepindolol, epanololl, tertatolol, nebivolol, esatenolol, asenapine, propafenone, levobetaxolol, buflolmedil, dutasteride, finasteride, ziprasidone, thioridazine, flupentixol, promazine, trazodone, risperidone, propiomazine, trifluoperazine, nefazodone, methotrimeprazine, dronedarone, nicardipine, paliperidone, quetiapine, clozapine, aripiprazole, olanzapine, droperidol, zuclopenthixol, amitriptyline, doxepin, imipramine, nortriptyline, amoxapine, trimipramine, chlorpromazine, acepromazine, thioproperaine, iloperidone, niguldipine, verapamil, pizotifen, propiverine, periciazine, brexpiprazole, bromocriptine, anisodamine, ergotamine, aripiprazole lauroxil, dexpropranolol, guanadrel, guanethidine, orm-12741, dihydroergotoxine, viloxazine, and any combination thereof

8. The method of claim 1, wherein the modulator of an adrenergic receptor is an agonist selected from the group consisting of droxidopa, pseudoephedrine, ephedrine, dipivefrin, midodrine, isoetharine, norepinephrine, phenylephrine, phenylpropanolamine, brimonidine, clonidine, metaraminol, guanabenz, dexmedetomidine, epinephrine, tizanidine, methoxamine, orciprenaline, dobutamine, ritodrine, terbutaline, bitolterol, oxymetazoline, salmeterol, apraclonidine, mehyldopa, formoterol, salbutamol, guanfacine, isoprenaline, arbutamine, arformoterol, fenoterol, pirbuterol, mephentermine, procaterol, clenbuterol, nebivolol, lofexidine, amibegro, nylidrin, solabegron, naphazoline, mirabegron, adrafinil, isoxsuprine, hexoprenaline, etilefrine, befunolol, olodaterol, cirazoline, synephrine, racepinephrine, amitraz, medetomidine, xylazine, ractopamine, romofidine, detomidine, rilmenidine, ritobegron, tulobuterol, dopexamine, higenamine, reproterol, octopamine, norfenefrine, oxyfedrine, rimiterol, methoxyphenamine, tretoquinol, prenalterol, xamoterol, ephedra sinica root, cl-methylephedrine, xylometazoline, pergolide, bromocriptine, metamfetamine, moxonidine, phendimetrozine, ergometrine, isometheptene, tetryzoline, etomidate, bambuterol, indacaterol, vilanterol, celiprolol, levosalbutamol, doxofylline, protokylol, etafedrine, bethanidine, abediterol, PF-00610355, anisodamine, hydroxyamphetamine, benzaphetamine, 4-methoxyamphetamine, droxidopa, and any combination thereof.

9. The method of claim 1, wherein the modulator of arginine vasopressin receptor is an antagonist selected from the group consisting of conivaptan, tolvaptan, lixivaptan, satavaptan, relcovaptan, nelivaptan, lixivaptan, mozavaptan, somatostatin, balovaptan, and any combination thereof.

10. The method of claim 1, wherein the modulator of arginine vasopressin receptor is an agonist selected from the group consisting of selepressin, terlipressin, and combinations thereof.

11. The method of claim 1, wherein the modulator of an adrenergic receptor is phenylephrine, phentolamine, norepinephrine 5-methylurapidil, L-786314, BMY7378, or any combination thereof.

12. The method of claim 1, further comprising administering a therapeutically effective amount of a cytokine receptor modulator.

13. The method of claim 12, wherein the cytokine receptor modulator is selected from the group consisting of maraviroc, plerixafor, vicriviroc, aplaviroc, BX471, CP-481, 715, MK-0812, T-487 (AMG-487), ZK-756326, IL-8, VUF 11207, Rh-SDF1α, AMD3100, and any combination thereof.

14. The method of claim 12, wherein the cytokine receptor modulator is a catecholamine.

15. The method of claim 12, wherein the therapeutically effective amount of cytokine receptor modulator is less than a therapeutically effective amount of cytokine receptor modulator when no modulator of an adrenergic receptor or an arginine vasopressin receptor is administered to the subject.

16. The method of claim 1, wherein the inflammation is from infection, trauma, autoimmune disease, cardiovascular disease, or cancer.

17. A composition comprising a modulator of an adrenergic receptor and/or an arginine vasopressin receptor, and at least one cytokine receptor modulator.
